(19) United States
(12) Patent Application Publication
(10) Pub. No.: US 2024/0209011 A1
(43) Pub. Date: Jun. 27, 2024

(54) HETEROAROMATIC PHOSPHONIUM SALTS FOR TREATING CANCER

(71) Applicant: FLORATEK PHARMA S.A., Aubonne (CH)

(72) Inventor: Dan Florin Stoicescu, Aubonne (CH)

(21) Appl. No.: 18/274,324

(22) PCT Filed: Jan. 26, 2022
(86) PCT No.: PCT/EP2022/051809

§ 371 Date: Jul. 26, 2023

(30) Foreign Application Priority Data
Jan. 26, 2021 (GB) 2101043.4

(51) Int. Cl. C07F 9/655; A61K 31/665; A61P 35/00

(52) U.S. Cl. CPC C07F 9/65522; A61K 31/665; A61P 35/00

(57) ABSTRACT

The present invention relates to chromen-4-one derivatives comprising a phosphonium quaternary group, and to associated multi-salts, solvates, prodrugs and pharmaceutical compositions. The present invention also relates to the use of such compounds and compositions in the treatment and prevention of cancer.

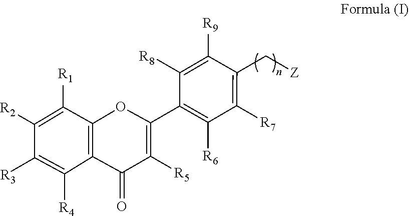

### FIELD OF THE INVENTION

The present invention relates to flavonoid compounds, and to associated multi-salts, solvates, prodrugs and pharmaceutical compositions. The present invention also relates to the use of such compounds and compositions in the treatment and prevention of cancer.

### BACKGROUND

Targeting delayed or inhibited apoptosis is a major approach in cancer treatment and a highly active area of research. Apoptosis is a stringently organized process, regulated by a series of signal transduction cascades and cellular proteins. Two major pathways contributing to apoptosis: firstly, the extrinsic/death receptor induced pathway and secondly, the intrinsic pathway in which mitochondrial stress is involved [Rathore R., McCallum J. E., Varghese E., Maria A., Busselberg D. Overcoming chemotherapy drug resistance by targeting inhibitors of apoptosis proteins (iaps) Apoptosis. 2017; 22:898-919]. Mitochondrial pathway of apoptosis is the most commonly deregulated type of cell death in cancer, and the understanding of mitochondrial apoptosis had advanced, so that novel therapies can be developed to specifically activate this process. [Lopez J., Tait S. W. G. Mitochondrial apoptosis: Killing cancer using the enemy within. Br. J. Cancer. 2015; 112:957-962]. In healthy cells, mitochondria execute a controlled regulation of multiple functions to maintain the cellular growth-death cycle. However, in the case of tumour cells, to meet the higher metabolic demand of rapidly proliferating cells, dysregulation of mitochondrial metabolism occurs. The difference between cancer cell mitochondria and normal cells includes several functional alterations, such as mutation of mtDNA, deficient respiration and ATP generation, mutation of mtDNA-encoded mitochondrial enzymes and structural differences, such as higher membrane potential of cancer cell mitochondria and higher basicity inside the mitochondrial lumen. The evasion of cell death or inhibition of mitochondria-mediated apoptosis is a hallmark for cancer. Mitochondria generate ROS, which is necessary for signalling under normal conditions. However, when apoptosis is inhibited in the case of cancer, ROS contributes to the neoplastic transformation. This altered mitochondrial metabolism of cancer cells compared with that of their normal counterparts is advantageous for the selective targeting of cancer mitochondria in therapeutics, which focuses on the cancer mitochondria specific features [Rin Jean S., Tulumello D. V., Wisnovsky S. P., Lei E. K., Pereira M. P., Kelley S. O. Molecular vehicles for mitochondrial chemical biology and drug delivery. ACS Chem. Biol. 2014; 9:323-333]. Anticancer drugs that selectively disrupt cancerous mitochondria could be achieved by designing molecules that act on the malignant mitochondria by, for instance, inhibiting glycolysis, depolarizing the membrane potential, and inhibiting the mitochondrial permeability transition pore [Dilip A., Cheng G., Joseph J., Kunnimalaiyaan S., Kalyanaraman B., Kunnimalaiyaan M., Gamblin T. C. Mitochondria-targeted antioxidant and glycolysis inhibition: Synergistic therapy in hepatocellular carcinoma. Anticancer Drugs. 2013; 24:881-888].

There is a need to provide compounds with improved pharmacological and/or physiological and/or physiochemical properties and/or those that provide a useful alternative to known compounds.

### SUMMARY OF THE INVENTION

The present invention addresses the limitations of the polyphenol class of compounds in maximizing their natural anti-cancer potential by providing a series of structurally novel compounds targeted to the mitochondrial membrane, thus enhancing the apoptotic pathway and potentially overcoming drug resistance by bypassing the cells mechanism of evading the apoptotic pathway. The compounds are effective through a multi-targeted approach using the lipophilic ion to rapidly penetrate and accumulate in the mitochondrial membrane and the polyphenolic moiety to exert anti-oxidant and antiproliferative effects. Additionally or alternatively, the discovered compound series optimizes the alkyl linker used to connect the lipophilic ion with the biologically active moiety.

The present invention is defined in the claims.

A first aspect of the invention provides a compound of formula (I) for use treating or preventing cancer:

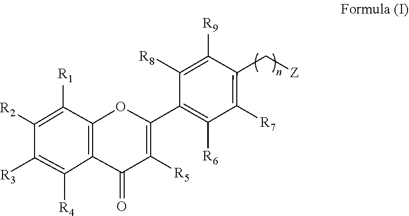

wherein:
Z is —[P(R¹¹)₃]X, wherein X is a counter anion;
R¹ and R², independently, are selected from —OH, —O—C₁₋₄ alkyl, —OC(O)R₁₃, —OC(O)NHR¹³, —OC(O)N(R¹³)₂; or R¹ and R² together form —O—(C₁₋₃ alkylene)-O—; R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(RP)₂; —NH₂; —NHR^{β}; —N(R^{β})₂; —CHO; —COR^{β}; —COOH; —COOR^{β}; —OCOR^{β}; and benzyl optionally substituted with 1-3-R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-10.

For example, n may be selected from an integer from 3 to 6.

For example, the compound may be a compound of Formula 1A:

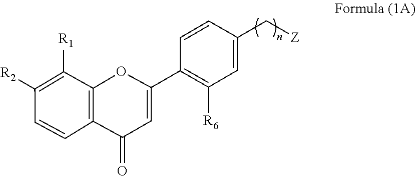

wherein:
Z is —[P(R¹¹)₃]X, wherein X is a counter anion;
R¹ and R², independently, are selected from —OH, —O—C₁₋₄ alkyl, —OC(O)R₁₃, —OC(O)NHR¹³, —OC(O)N(R¹³)₂; or R¹ and R² together form —O—(C₁₋₃ alkylene)-O—;
R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3-R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-10.

For example, n may be selected from an integer between 3 and 6.

A second aspect of the invention provides a compound selected from the following group:

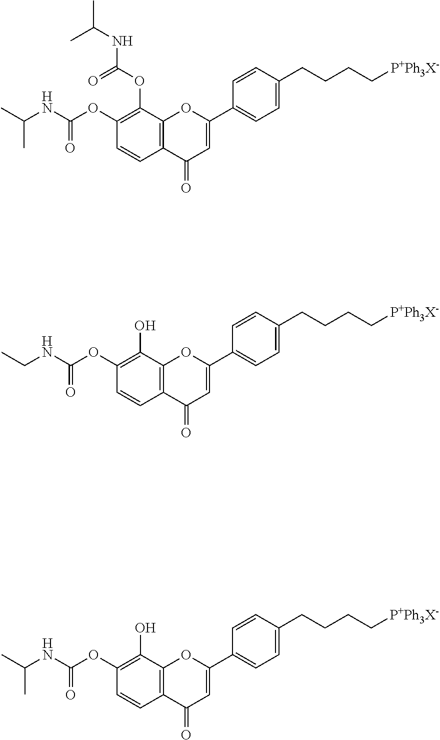

Wherein X is as defined herein.

A third aspect of the invention provides a pharmaceutically acceptable multi-salt, solvate or prodrug of the compound of the second aspect of the invention.

A fourth aspect of the invention provides a pharmaceutical composition comprising a compound of the second aspect of the invention, or a pharmaceutically acceptable multi-salt, solvate or prodrug of the third aspect of the invention, and a pharmaceutically acceptable excipient.

A fifth aspect of the invention provides a compound of the second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. In one embodiment, the disease, disorder or condition is cancer.

A sixth aspect of the invention provides the use of a compound of the second aspect, a pharmaceutically effective multi-salt, solvate or prodrug of the third aspect, or a pharmaceutical composition according to the fourth aspect, in the manufacture of a medicament for the treatment or prevention of a disease, disorder or condition. Typically the treatment or prevention comprises the administration of the compound, multi-salt, solvate, prodrug or pharmaceutical composition to a subject. In one embodiment, the disease, disorder or condition is cancer.

A seventh aspect of the invention provides a method of treatment or prevention of a disease, disorder or condition, the method comprising the step of administering an effective amount of a compound of the second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof. In one embodiment, the disease, disorder or condition is cancer.

An eighth aspect of the invention provides a method of treatment or prevention of a disease, disorder or condition, the method comprising the step of administering an effective amount of a compound according to formula (1) as defined herein, or a pharmaceutically acceptable multi-salt, solvate or prodrug thereof, to thereby treat or prevent the disease, disorder or condition. Typically the administration is to a subject in need thereof. In one embodiment, the disease, disorder or condition is cancer.

### BRIEF DESCRIPTION OF THE FIGURES

FIG. 1 shows an IC50 curve of SND118 (Cpd A) against brain carcinoma cell line U-87.

FIG. 2 shows an IC50 curve of SND124 (Cpd B) against brain carcinoma cell line U-87.

FIG. 3 shows an IC50 curve of SND140 against a brain carcinoma PDX GBM14-CHA.

FIG. 4 shows an IC50 curve of SND118 (Cpd A) against breast carcinoma cell line MCF-7.

FIG. 5 shows an IC50 curve of SND118 (Cpd A) against breast carcinoma cell line MDA-MB-468.

FIG. 6 shows an IC50 curve of SND118 (Cpd A) against colon carcinoma cell line HCT116.

FIG. 7 shows an IC50 curve of SND124 (Cpd B) against colon carcinoma cell line HT-29.

FIG. 8 shows an IC50 curve of SND118 (Cpd A) against leukaemia cell line K-562.

FIG. 9 shows an IC50 curve of SND124 (Cpd B) against leukaemia cell line K-562.

FIG. 10 shows an IC50 curve of SND118 (Cpd A) against leukaemia cell line HL-60.

FIG. 11 shows IC50 curve of SND118 (Cpd A) against NSCLC cell line NCI-H-1299.

FIG. 12 shows IC50 curve of SND124 (Cpd B) against NSCLC cell line NCI-H-1299.

FIG. 13 shows IC50 curve of SND140 against small cell lung carcinoma PDX SC6 cell line.

FIG. 14 shows IC50 curve of SND118 (Cpd A) against ovarian cell line SK-OV-3.

FIG. 15 shows IC50 curve of SND124 (Cpd B) against pancreatic cell line Mia-Pa-Ca-2.

FIG. 16 shows IC50 curve of SND118 (Cpd A) against prostate cell line LNCaP.

### DETAILED DESCRIPTION OF THE INVENTION

A first aspect of the invention provides a compound of formula (I) for use treating or preventing cancer:

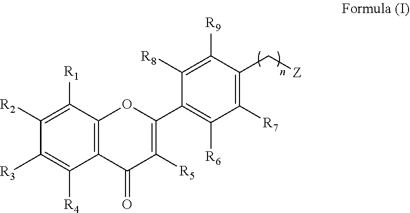

wherein:
Z is —[P(R¹¹)₃]X, wherein X is a counter anion;
R¹ and R², independently, are selected from —OH, —O—C₁₋₄ alkyl, —OC(O)R₁₃, —OC(O)NHR¹³, —OC(O)N(R¹³)₂; or R¹ and R² together form —O—(C₁₋₃ alkylene)-O—;
R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(RP)₂; —NH₂; —NHR^{β}; —N(R^{β})₂; —CHO; —COR^{β}; —COOH; —COOR^{β}; —OCOR^{β}; and benzyl optionally substituted with 1-3-R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups;
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl;
n=1-10.

In one embodiment, n=3-6.

In one embodiment, n is 3, 4, 5 or 6.

In one embodiment, n is 3 or 4.

In one embodiment, R¹ and R², independently, are selected from —OH, —O—C₁₋₄ alkyl, —OC(O)R₁₃, —OC(O)NHR¹³, and —OC(O)N(R¹³)₂; or R¹ and R² together form —O—(C₁₋₃ alkylene)-O—.

In one embodiment, R¹ and R², independently, are selected from —OH, —O—C₁₋₄ alkyl, —OC(O)R₁₃, —OC(O)NHR¹³, and —OC(O)N(R¹³)₂.

In one embodiment, R¹ and R², independently, are selected from —OH, —OCH₃, —OC(O)C(CH₃)₃, —OC(O)NH—C₁₋₃ alkyl, and —OC(O)N(CH₃)₂, or R¹ and R² together form —O—CH₂—O—.

In one embodiment, R¹ and R², independently, are selected from —OH, —OCH₃, —OC(O)C(CH₃)₃, —OC(O)NH—C₁₋₃ alkyl, and —OC(O)N(CH₃)₂.

In one embodiment, R¹ and R² together form a —O—(C₁₋₃ alkylene)-O— group. For example, R¹ and R² together form —O-(methylene)-O—.

In one embodiment, R¹ is —OH, and R² is selected from —OH, —O—C₁₋₄ alkyl, —OC(O)R₁₃, —OC(O)NHR¹³, and —OC(O)N(R¹³)₂. For example, R¹ is —OH, and R² is selected from —OH, —OC(O)R₁₃, —OC(O)NHR¹³, and —OC(O)N(R¹³)₂. For example, R¹ is —OH, and R² is selected from —OH, —OC(O)—C₃₋₄-alkyl; —OC(O)NH—C₂₋₃-alkyl, and —OC(O)N(—C₂₋₃-alkyl)₂. For example, R¹ is —OH, and R² is selected from —OH, —OC(O)—C₄-alkyl; —OC(O)NH—C₂₋₃-alkyl, and —OC(O)N(—C₂₋₃-alkyl)₂.

In one embodiment, R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; —COOR^{β}; —OCOR^{β}; and benzyl optionally substituted with 1-3-R^{β}. In one embodiment, R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; and —COOR^{β}; and benzyl optionally substituted with 1-3-R^{β}. In one embodiment, R³, R⁴, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; —COOR^{β}; and —OCOR^{β}. In one embodiment, R³, R⁴, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; and —COOR^{β}. In one embodiment, R³, R⁴, R⁵, R⁶, R⁷, R⁸, and R⁹, independently, are selected from H; halo; —CN; —NO₂; —R^{β}; —NH₂; —NHR^{β}; —N(R^{β})₂; and —CHO. In one embodiment, R³, R⁴, R⁵, R⁶, R⁷, R⁸, and R⁹, independently, are selected from H; halo; —CN; —NO₂; —SH; —SO₂H; and —NH₂. In one embodiment, R³, R⁴, R⁵, R⁶, R⁷, R⁸, and R⁹ are H.

In one embodiment, R¹ and R², independently, are selected from —OH, —O—C₁₋₄ alkyl, —OC(O)R₁₃, —OC(O)NHR¹³, and —OC(O)N(R¹³)₂; or R¹ and R² together form —O—(C₁₋₃ alkylene)-O—; and R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(RP)₂; —NH₂; —NHR^{β}; —N(R^{β})₂; —CHO; —COR^{β}; —COOH; —COOR^{β}; —OCOR^{β}; and benzyl optionally substituted with 1-3-R^{β}.

In one embodiment, R¹ and R², independently, are selected from —OH, —O—C₁₋₄ alkyl, —OC(O)R₁₃, —OC(O)NHR¹³, and —OC(O)N(R¹³)₂; and R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(RP)₂; —NH₂; —NHR^{β}; —N(R^{β})₂; —CHO; —COR^{β}; —COOH; —COOR^{β}; —OCOR^{β}; and benzyl optionally substituted with 1-3-R^{β}.

In one embodiment, R¹ and R², independently, are selected from —OH, —O—C₁₋₄ alkyl, —OC(O)R₁₃, —OC(O)NHR¹³, and —OC(O)N(R¹³)₂, or R¹ and R² together form a —O—(C₁₋₃ alkylene)-O— group; and R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(RP)₂; —NH₂; —NHR^{β}; —N(R^{β})₂; —CHO; —COR^{β}; —COOH; —COOR^{β}; —OCOR^{β}; and benzyl optionally substituted with 1-3-R^{β}. For example, R¹, and R², independently, are selected from —OH, —OCH₃, —OC(O)C(CH₃)₃, —OC(O)NH—C₁₋₃ alkyl, and —OC(O)N(CH₃)₂; and R³, R⁴, R⁵, R⁶, R⁷, R⁸, and R⁹, independently, are selected from H; halo; —CN; —NO₂; —SH; —SO₂H; and —NH₂. For example, R¹, and R², independently, are selected from —OH, —OCH₃, —OC(O)C(CH₃)₃, —OC(O)NH—C₁₋₃ alkyl, and —OC(O)N(CH₃)₂; and R³, R⁴, R⁵, R⁶, R⁷, R⁸, and R⁹ are H.

In one embodiment, R¹ and R², independently, are selected from —OH and —O—C₁₋₄ alkyl, or R¹ and R² together form a —O—(C₁₋₃ alkylene)-O— group; and R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(RP)₂; —NH₂; —NHR^{β}; —N(R^{β})₂; —CHO; —COR^{β}; —COOH; —COOR^{β}; —OCOR^{β}; and benzyl optionally substituted with 1-3-R^{β}. For example, R¹ and R², independently, are selected from —OH, and —OCH₃; and R³, R⁴, R⁵, R⁶, R⁷, R⁸, and R⁹, independently, are selected from H; halo; —CN; —NO₂; —SH; —SO₂H; and —NH₂. For example, R¹ and R², independently, are selected from —OH, and —OCH₃; and R³, R⁴, R⁵, R⁶, R⁷, R⁸, and R⁹ are H.

In one embodiment, each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups.

In one embodiment, R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more halo, —OH, —NH₂, —CN, —NO₂, —C≡CH, —CHO, —CON(CH₃)₂ or oxo (═O) groups.

In one embodiment, each —R^{β} is independently selected from a C₁-C₆ alkyl, C₂-C₆ alkenyl, C₂-C₆ alkynyl or C₃-C₁₄ cyclic group.

In one embodiment, each —R^{β} is independently selected from —CF₃ and —CHF₂.

In one embodiment, each —R^{β} is independently selected from a methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl group.

In one embodiment, each —R^{β} is independently selected from a methyl, ethyl, n-propyl, i-propyl, n-butyl, i-butyl, t-butyl, or n-pentyl group.

X is a pharmaceutically acceptable counter anion. In one embodiment, X is selected from but not limited to halides (for example fluoride, chloride, bromide or iodide) or other inorganic anions (for example nitrate, perchlorate, sulfate, bisulfate, or phosphate) or organic anions (for example propianoate, butyrate, glycolate, lactate, mandelate, citrate, acetate, benzoate, salicylate, succinate, malate, tartrate, fumarate, maleate, hydroxymaleate, galactarate, gluconate, pantothenate, pamoate, methanesulfonate, trifluoromethanesulfonare, ethanesulfonare, 2-hydroxyethanesulfonate, benzenesulfonate, toluene-p-sulfonate, naphthalene-2-sulfonate, camphorsulfonate, ornithinate, glutamate or aspartate).

In one embodiment, X may be a fluoride, chloride, bromide or iodide.

In one embodiment, X is bromide or chloride.

In one embodiment, X is bromide or iodide.

In one embodiment, X is bromide.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion.

For example, X may be bromide, iodide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide, iodide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups; and wherein X is a counter anion.

For example, X may be bromide, iodide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide, iodide or chloride.

In one embodiment, Z is —[P(R¹¹)₃]X, wherein each —R¹¹ is independently selected from H, C₁-C₆ alkyl, C₂-C₆ alkenyl, C₃-C₁₄ aryl group, or C₃-C₁₄ aliphatic cyclic group; and wherein X is a counter anion. For example, X may be bromide, iodide or chloride.

In one embodiment, Z is —[P(R¹¹)₃]X, wherein each —R¹¹ is independently selected from H, or C₁-C₆ alkyl, or C₃-C₁₄ aryl group; and wherein X is a counter anion. For example, X may be bromide, iodide or chloride.

In one embodiment, Z is —[P(R¹¹)₃]X, wherein each —R¹¹ is independently a C₃-C₁₄ aryl group; and wherein any —R¹¹ may optionally be substituted with one or more C₁-C₄ alkyl, halo, —OH, —NH₂, —CN, —C≡CH or oxo (═O) groups; and wherein X is a counter anion. For example, X may be bromide, iodide or chloride.

In one embodiment, two of the R¹¹ groups are the same. In one embodiment, each R¹¹ group is the same.

In one embodiment, each Ru group is the same; preferably each R¹¹ is a phenyl group.

In one embodiment, Z is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; and wherein X is a counter anion. For example, X may be bromide, iodide or chloride.

In one embodiment, each R¹¹ is a phenyl group.

In one embodiment, Z is —[P(Ph)₃]X, wherein X is a counter anion. For example, X may be bromide or chloride, or X may be bromide.

In one embodiment, each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴.

In one embodiment, each —R¹³ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl.

In one embodiment, each —R¹³ is independently selected from C₁₋₄ alkyl. For example, R¹³ is independently selected from C₁₋₃ alkyl.

In one embodiment, each —R¹³ is independently selected from a H, methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl group.

In one embodiment, each —R¹³ is independently selected from H, methyl, ethyl, n-propyl, i-propyl, n-butyl, i-butyl, t-butyl, or n-pentyl group.

In one embodiment, each —R¹³ is independently selected from H, methyl, ethyl, propyl, and butyl.

In one embodiment, each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅.

In one embodiment, each R¹⁴ is independently selected from a halo, —NO₂, —CN, —OH, —NH₂, mercapto, formyl, carboxy, or carbamoyl group.

In one embodiment, each —R¹⁴ is independently selected from methyl, ethyl, n-propyl, i-propyl, n-butyl, i-butyl, t-butyl, n-pentyl, ethenyl, propenyl, 1-butenyl, 2-butenyl, 1-pentenyl, 1-hexenyl, 1,3-butadienyl, 1,3-pentadienyl, 1,4-pentadienyl, 1,4-hexadienyl, ethynyl, propargyl, but-1-ynyl or but-2-ynyl.

In one embodiment, each —R¹⁴ is independently selected from a methyl, ethyl, n-propyl, i-propyl, n-butyl, i-butyl, t-butyl, or n-pentyl group.

In one embodiment, each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl.

In one embodiment, n is an integer from 3 to 5. In one embodiment, n is an integer from 4 to 6. In one embodiment, n is 3, 4, 5, or 6. In one embodiment, n is 3. In one embodiment, n is 4.

In one embodiment, R¹ and R² are independently selected from —OH, —OCH₃, —OCO^{t}Bu, —OCONHCH₃, —OCONHCH₂CH₃ and —OCON(CH₃)₂, or R¹ and R² together form —O—CH₂—O—; R³, R⁴, R⁵, R⁶, R⁷, R⁸, and R⁹ are each H; Z is —[P(R¹¹)₃]X, wherein each —R¹¹ is a phenyl group; each phenyl group may optionally be substituted with one or more C₁-C₄ alkyl, halo, —OH, —NH₂, —CN, —C≡CH or oxo (═O) groups; X is a counter anion; and n is 3 or 4. For example, X may be bromide or chloride, or X may be bromide.

In one embodiment, R¹ and R² are independently selected from —OH, —OCH₃, —OCO^{t}Bu, —OCONHCH₃, —OCONHCH₂CH₃ or —OCON(CH₃)₂, or R¹ and R² together form —O—CH₂—O—; R³, R⁴, R⁵, R⁶, R⁷, R⁸, and R⁹ are each H; Z is —[P(Ph)₃]X; X is a counter anion; and n is 3 or 4. For example, X may be bromide or chloride, or X may be bromide.

In one embodiment, the compounds include a quaternary phosphonium group and X is a counter anion. Preferably, the counter anion X may be any pharmaceutically acceptable, non-toxic counter ion. For example, X may be bromide or chloride, or X may be bromide.

The counter anion may optionally be singly, doubly or triply charged. As the quaternary group is singly charged, if the counter anion is triply charged then the stoichiometric ratio of the quaternary group to counter anion will typically be 3:1 and if the counter anion is doubly charged then the stoichiometric ratio of the quaternary group to counter anion will typically be 2:1. If both the quaternary group and the counter anion are singly charged then the stoichiometric ratio of the quaternary group to counter anion will typically be 1:1.

In one embodiment, the counter anion will be a singly charged anion. Suitable anions X include but are not limited to halides (for example fluoride, chloride, bromide or iodide) or other inorganic anions (for example nitrate, perchlorate, sulfate, bisulfate, or phosphate) or organic anions (for example propianoate, butyrate, glycolate, lactate, mandelate, citrate, acetate, benzoate, salicylate, succinate, malate, tartrate, fumarate, maleate, hydroxymaleate, galactarate, gluconate, pantothenate, pamoate, methanesulfonate, trifluoromethanesulfonare, ethanesulfonare, 2-hydroxyethanesulfonate, benzenesulfonate, toluene-p-sulfonate, naphthalene-2-sulfonate, camphorsulfonate, ornithinate, glutamate or aspartate). The counter anion may be fluoride, chloride, bromide or iodide. For example, X may be bromide or chloride, or X may be bromide.

In one embodiment, R³, R⁴, R⁵, R⁷, R⁸, and R⁹ are H; and R⁶ is selected from —OH, —O—C₁₋₄ alkyl, —OC(O)R₁₃, —OC(O)NHR¹³, —OC(O)N(R¹³)₂. This corresponds to a compound of formula (1A):

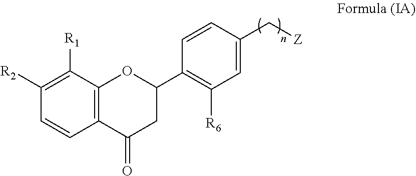

wherein R¹, R², R⁶ and Z are as defined herein.

In one aspect of any of the above embodiments, the compound of formula (I) has a molecular weight of from 250 to 2,000 Da. Typically, the compound of formula (I) has a molecular weight of from 300 to 1,000 Da. Typically, the compound of formula (I) has a molecular weight of from 350 to 800 Da. More typically, the compound of formula (I) has a molecular weight of from 500 to 750 Da.

In one embodiment, the compound is selected from the group consisting of:

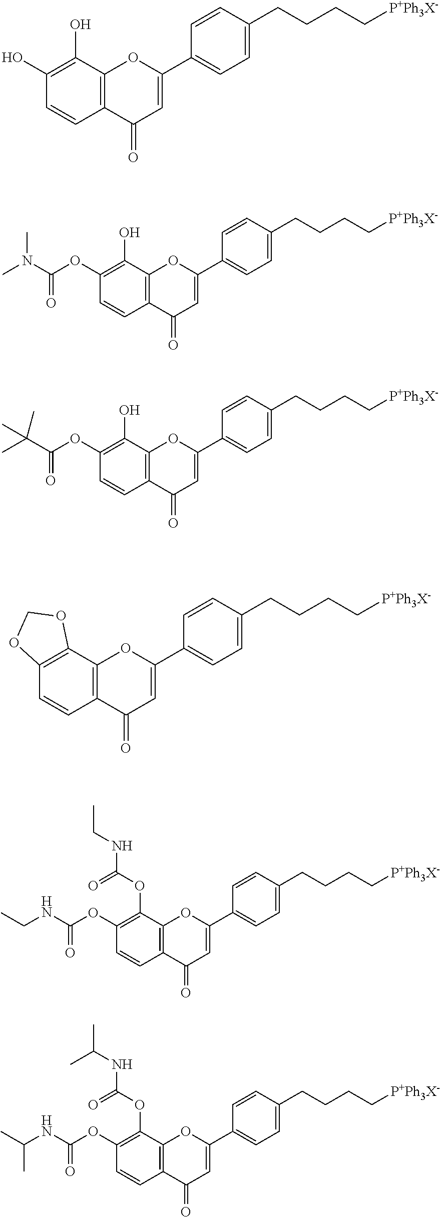

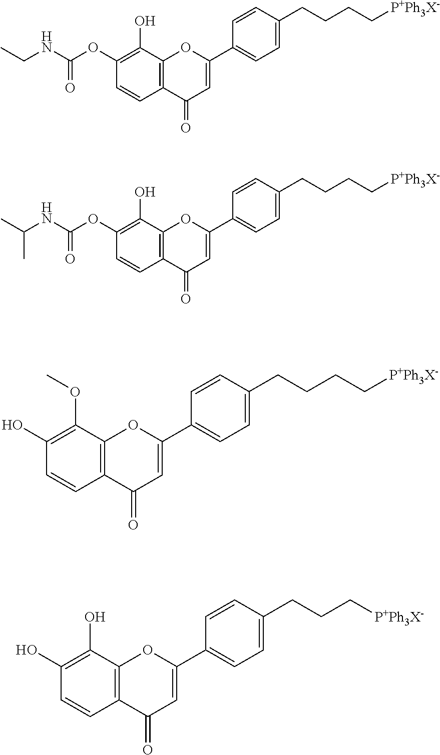

In one embodiment, the compound is selected from the group consisting of:

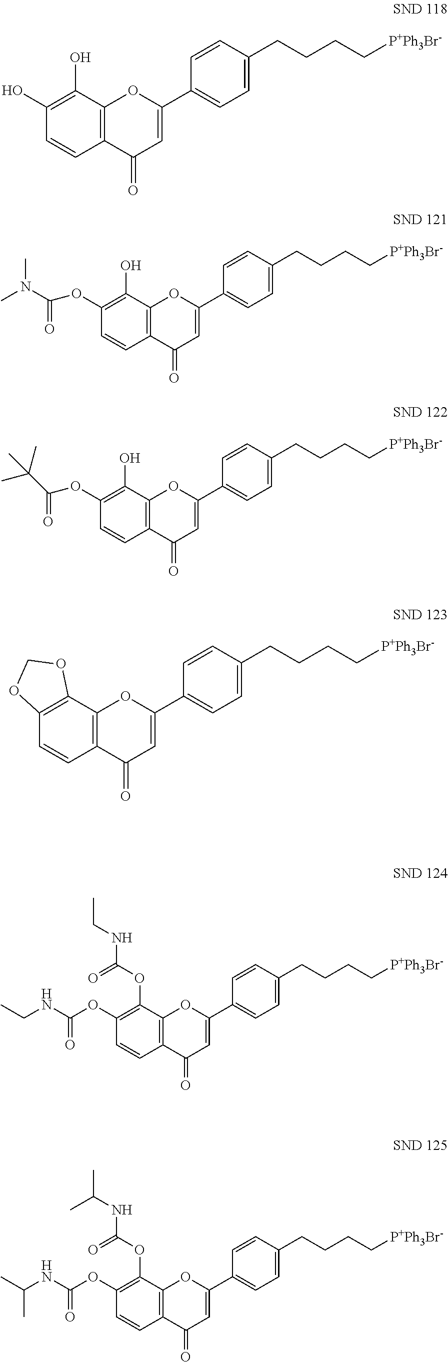

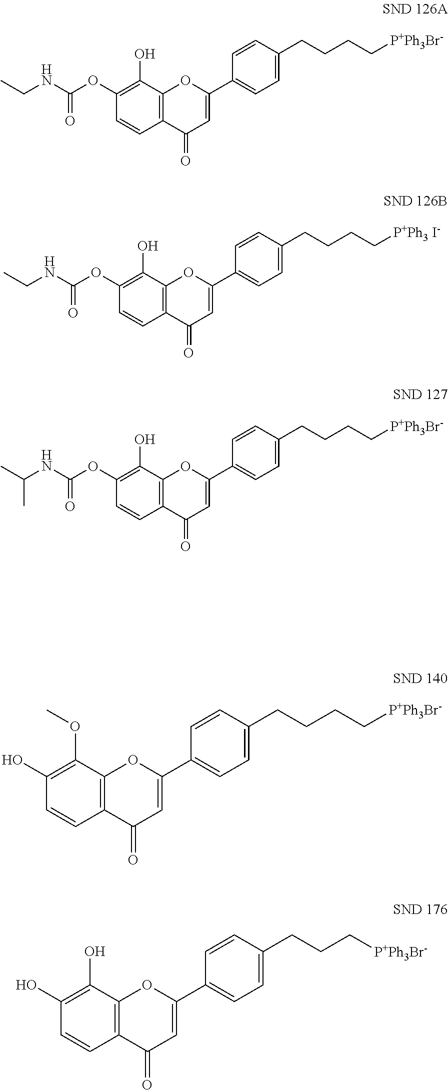

In one embodiment, the compound is selected from the group consisting of:

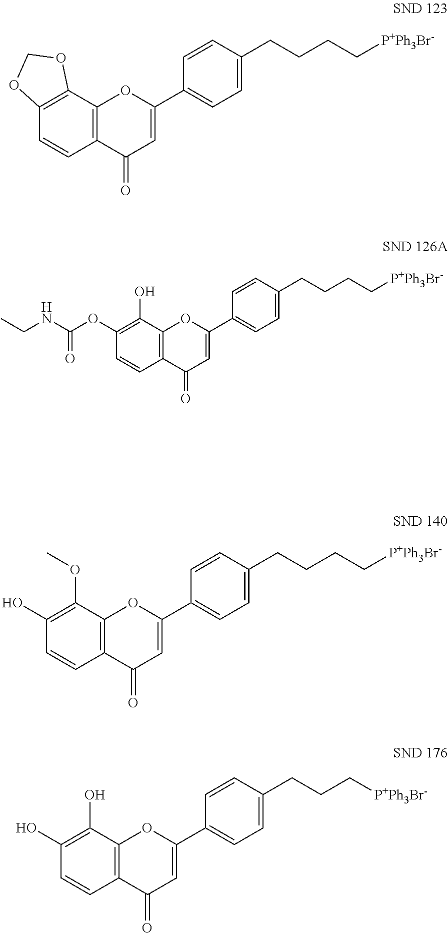

A second aspect of the invention provides a compound selected from the following group of compounds:

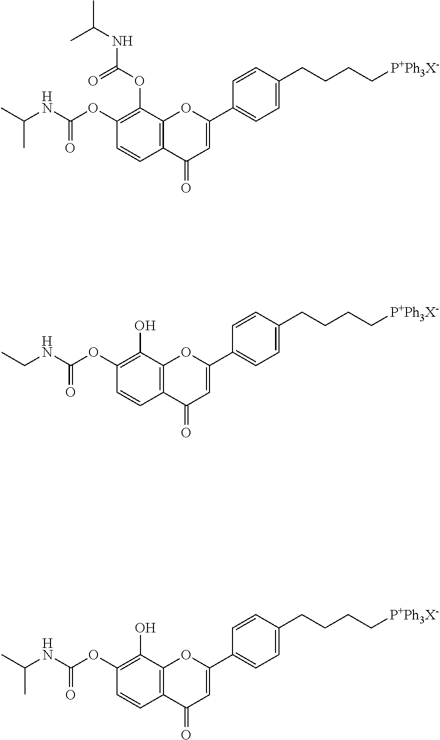

Wherein X is as defined herein.

In one embodiment, the compounds are selected from:

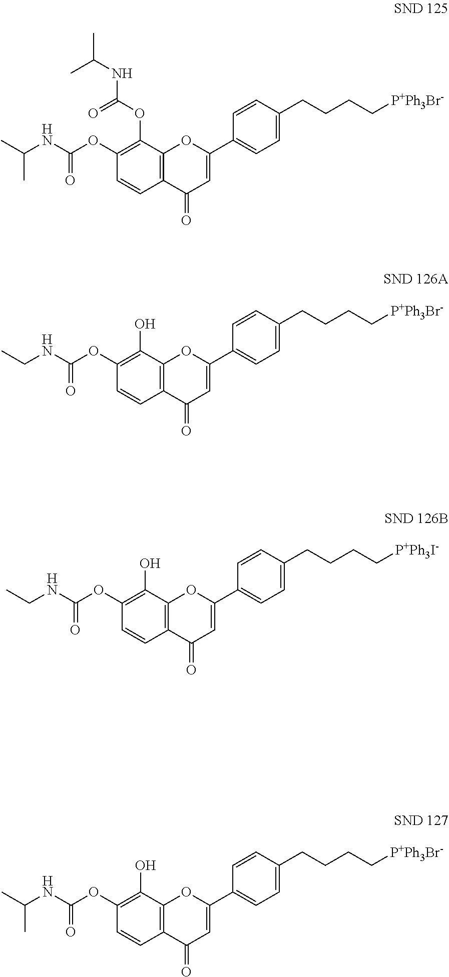

A third aspect of the invention provides a pharmaceutically acceptable multi-salt, solvate or prodrug of any compound of the second aspect of the invention.

The compounds of the present invention can be used both in their quaternary salt form (as a single salt). Additionally, the compounds of the present invention may contain one or more (e.g. one or two) acid addition or alkali addition salts to form a multi-salt. A multi-salt includes a quaternary salt group as well as a salt of a different group of the compound of the invention.

For the purposes of this invention, a “multi-salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

The compounds of the present invention can be used both, in quaternary salt form and their multi-salt form. For the purposes of this invention, a “multi-salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt.

Preferably the salt is a mono- or di-lithium, sodium, potassium, magnesium, calcium or ammonium salt. More preferably the salt is a mono- or di-sodium salt or a mono- or di-potassium salt.

Preferably any multi-salt is a pharmaceutically acceptable non-toxic salt. However, in addition to pharmaceutically acceptable multi-salts, other salts are included in the present invention, since they have potential to serve as intermediates in the purification or preparation of other, for example, pharmaceutically acceptable salts, or are useful for identification, characterisation or purification of the free acid or base.

The compounds and/or multi-salts of the present invention may be anhydrous or in the form of a hydrate (e.g. a hemihydrate, monohydrate, dihydrate or trihydrate) or other solvate. Such solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound.

Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses multi-salts and solvates of such prodrugs as described above.

The compounds, multi-salts, solvates and prodrugs of the present invention may contain at least one chiral centre. The compounds, multi-salts, solvates and prodrugs may therefore exist in at least two isomeric forms. The present invention encompasses racemic mixtures of the compounds, multi-salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, multi-salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, multi-salts, solvates and prodrugs of the present invention may be in any polymorphic or amorphous form.

A fourth aspect of the invention provides a pharmaceutical composition comprising a compound of the second aspect of the invention, or a pharmaceutically acceptable multi-salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

A fifth aspect of the invention provides a compound of the first or second aspect of the invention, or a pharmaceutically acceptable multi-salt, solvate or prodrug of the third aspect of the invention, or a pharmaceutical composition of the fourth aspect of the invention, for use in medicine, and/or for use in the treatment or prevention of a disease, disorder or condition. Typically the use comprises the administration of the compound, multi-salt, solvate, prodrug or pharmaceutical composition to a subject.

An sixth aspect of the invention provides the use of a compound of the first or second aspect, a pharmaceutically effective multi-salt, solvate or prodrug of the third aspect, or a pharmaceutical composition according to the fourth aspect in the manufacture of a medicament for the treatment or prevention of a disease, disorder or condition. Typically the treatment or prevention comprises the administration of the compound, multi-salt, solvate, prodrug or pharmaceutical composition to a subject.

A seventh aspect of the invention provides a method of treatment or prevention of a disease, disorder or condition, the method comprising the step of administering an effective amount of a compound of the first or second aspect, or a pharmaceutically acceptable multi-salt, solvate or prodrug of the third aspect, or a pharmaceutical composition of the fourth aspect, to thereby treat or prevent the disease, disorder or condition. Typically the administration is to a subject in need thereof.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, multi-salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, beta-amyloid 42, tau and phosphor-tau.

In general embodiments, the disease, disorder or condition is cancer.

In one embodiment, the cancer is brain cancer, breast cancer, colon cancer, leukaemia, lung cancer, lymphoma, ovarian cancer, pancreatic cancer, prostate cancer, renal cancer or skin cancer (melanoma).

In one embodiment the cancer is brain cancer.

In one embodiment the cancer is breast cancer.

In one embodiment the cancer is colon cancer.

In one embodiment the cancer is leukaemia.

In one embodiment the cancer is lung cancer.

In one embodiment the cancer is lymphoma.

In one embodiment the cancer is ovarian cancer.

In one embodiment the cancer is pancreatic cancer.

In one embodiment the cancer is prostate cancer.

In one embodiment the cancer is renal cancer.

In one embodiment the cancer is skin cancer (melanoma)

Unless stated otherwise, in any aspect of the invention, the subject may be any human or other animal. Typically, the subject is a mammal, more typically a human or a domesticated mammal such as a cow, pig, lamb, goat, horse, cat, dog, etc. Most typically, the subject is a human.

Any of the medicaments employed in the present invention can be administered by oral, parental (including intravenous, subcutaneous, intramuscular, intradermal, intratracheal, intraperitoneal, intraarticular, intracranial and epidural), airway (aerosol), rectal, vaginal or topical (including transdermal, buccal, mucosal and sublingual) administration.

Typically, the mode of administration selected is that most appropriate to the disorder or disease to be treated or prevented.

For oral administration, the compounds, multi-salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or tale. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, multi-salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For transdermal and other topical administration, the compounds, multi-salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, multi-salts, solvates or prodrugs of the present invention will, of course, vary with the disorder or disease to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

An eighth aspect of the invention provides a method of treatment or prevention of a disease, disorder or condition, the method comprising the step of administering an effective amount of a compound according to formula (1) as defined herein, or a pharmaceutically acceptable multi-salt, solvate or prodrug thereof, to thereby treat or prevent the disease, disorder or condition. Typically the administration is to a subject in need thereof. In one embodiment, the disease, disorder or condition is cancer.

### Definitions

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds.

Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “haloalkyl” substituent group or haloalkyl group in a substituent group refers to an alkyl, alkenyl, or alkynyl substituent group or moiety including one or more carbon atoms and one or more halo atoms, e.g. Cl, Br, I, or F. Each halo atom replaces a hydrogen of the alkyl, alkenyl, or alkynyl substituent group or moiety. Examples include —CH₂F—CHF₂, —CHI₂, —CHBr₂, —CHCl₂, —CF₃, —CH₂CF₃ and CF₂CH₃.

An “alkoxy” substituent group or alkoxy group in a substituent group refers to an alkyl, alkenyl, or alkynyl substituent group or moiety including one or more carbon atoms and one or more oxygen atoms. Each oxygen atom replaces a carbon atom (for example the terminal or bonding carbon) of the alkyl, alkenyl, or alkynyl substituent group or moiety. Examples include —OCH₃, —OCH₂CH₃, —OCH₂CH₂CH₃, and —OCH(CH₃)(CH₃).

An “alkylthio” substituent group or alkylthio group in a substituent group refers to an alkyl, alkenyl, or alkynyl substituent group or moiety including one or more carbon atoms and one or more sulphur atoms. Each sulphur atom replaces a carbon atom (for example the terminal or bonding carbon) of the alkyl, alkenyl, or alkynyl substituent group or moiety. Examples include —SCH₃, —SCH₂CH₃, —SCH₂CH₂CH₃, and —SCH(CH₃)(CH₃).

An “alkylsulfinyl” substituent group or alkylsulfinyl group in a substituent group refers to an alkyl, alkenyl, or alkynyl substituent group or moiety including one or more carbon atoms and one or more sulfinyl groups (—S(═O)—). Each sulfinyl group replaces a carbon atom (for example the terminal or bonding carbon) of the alkyl, alkenyl, or alkynyl substituent group or moiety. Examples include —S(═O)CH₃, —S(═O)CH₂CH₃, —S(═O)CH₂CH₂CH₃, and —S(═O)CH(CH₃)(CH₃).

An “alkylsulfonyl” substituent group or alkylsulfonyl group in a substituent group refers to an alkyl, alkenyl, or alkynyl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

An “arylsulfonyl” substituent group or arylsulfonyl group in a substituent group refers to an aryl substituent group or moiety including one or more carbon atoms and one or more sulfonyl groups (—SO₂—). Each sulfonyl group replaces a carbon atom (for example the terminal or bonding carbon) of the alkyl, alkenyl, or alkynyl substituent group or moiety. Examples include —SO₂(CH₃), —SO₂(CH₂CH₃), —SO₂(CH₂CH₂CH₃), and —SO₂(CH(CH₃)(CH₃)).

A “cyclic” substituent group or a cyclic moiety in a substituent group refers to any hydrocarbyl ring, wherein the hydrocarbyl ring may be saturated or unsaturated and may include one or more heteroatoms, e.g. N, O or S, in its carbon skeleton. Examples of cyclic groups include aliphatic cyclic, cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more heteroatoms, e.g. N, O or S, in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl and thiomorpholinyl groups.

An “aliphatic cyclic” substituent group or aliphatic cyclic moiety in a substituent group refers to a hydrocarbyl cyclic group or moiety that is not aromatic. The aliphatic cyclic group may be saturated or unsaturated and may include one or more heteroatoms, e.g. N, O or S, in its carbon skeleton. Examples include cyclopropyl, cyclohexyl and morpholinyl. Unless stated otherwise, an aliphatic cyclic substituent group or moiety may include monocyclic, bicyclic or polycyclic hydrocarbyl rings.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

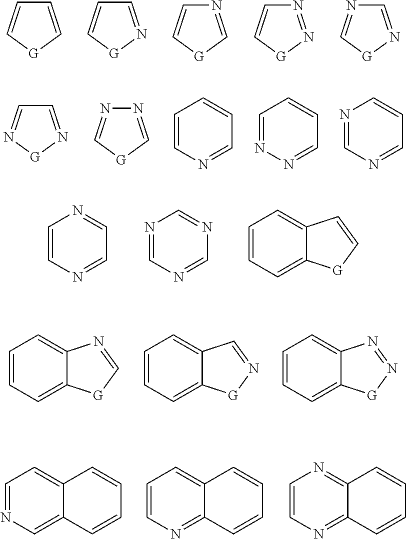

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

Typically a substituted group comprises 1, 2, 3 or 4 substituents, more typically 1, 2 or 3 substituents, more typically 1 or 2 substituents, and even more typically 1 substituent.

Unless stated otherwise, any divalent bridging substituent (e.g. —O—, —S—, —NH—, —N(R^{β})— or —R^{α}—) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Where reference is made to a carbon atom of a group being replaced by an N, O or S atom, what is intended is that:

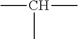

is replaced by

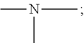

—CH₂— is replaced by —NH—, —O— or —S—;
—CH₃ is replaced by —NH₂, —OH, or —SH;
—CH═ is replaced by —N═;
CH₂═ is replaced by NH═, O═ or S═; or
CH≡ is replaced by N≡.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are counted as carbon atoms when calculating the number of carbon atoms in a Cₓ-C_{y} group. For example, a morpholinyl group is to be considered a C₆ heterocyclic group, not a C₄ heterocyclic group.

A “protecting group” refers to a grouping of atoms that when attached to a reactive functional group (e.g. OH) in a compound masks, reduces or prevents reactivity of the functional group.

In the context of the present specification, ═ is a double bond; ≡ is a triple bond.

The protection and deprotection of functional groups is described in ‘Protective Groups in Organic Synthesis’, 2^{nd} edition, T. W. Greene and P. G. M Wuts, Wiley-Interscience.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

The following nomenclature is used to refer to the following compounds.

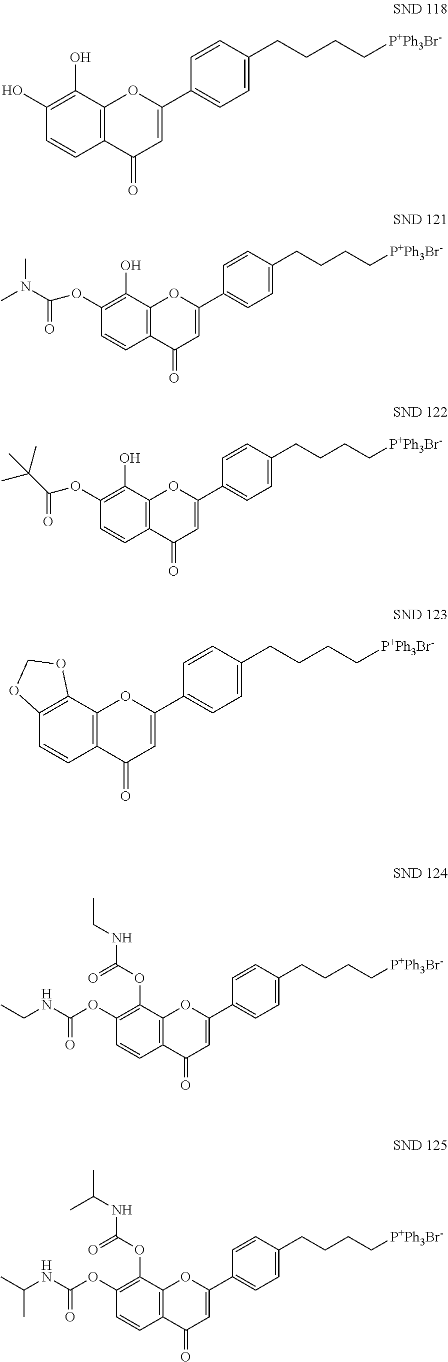

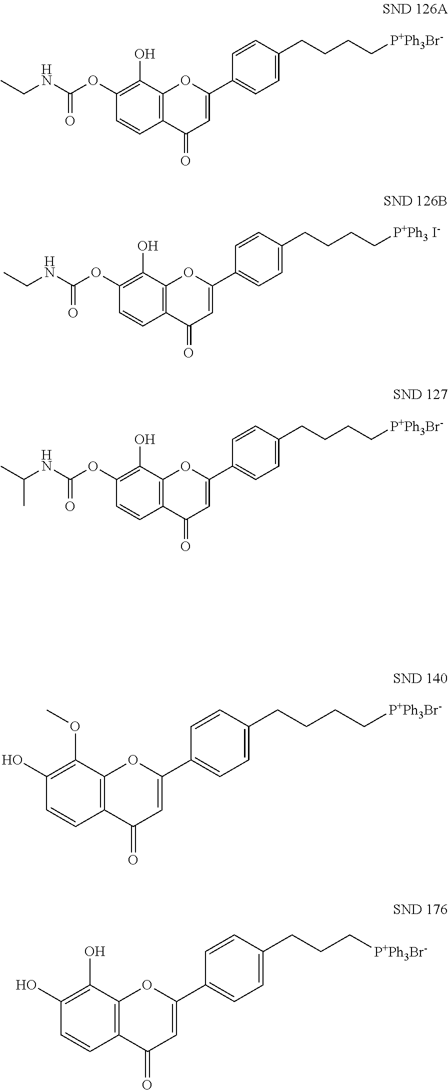

### Examples—Compound Synthesis

Compounds of the invention are synthesised employing a route of synthesis shown below. The general route of synthesis is illustrated below by reference to the synthesis of a specific compound. However, this is merely illustrative of a more general synthesis that can be employed to synthesise all compounds of the invention.

### Route of Synthesis:

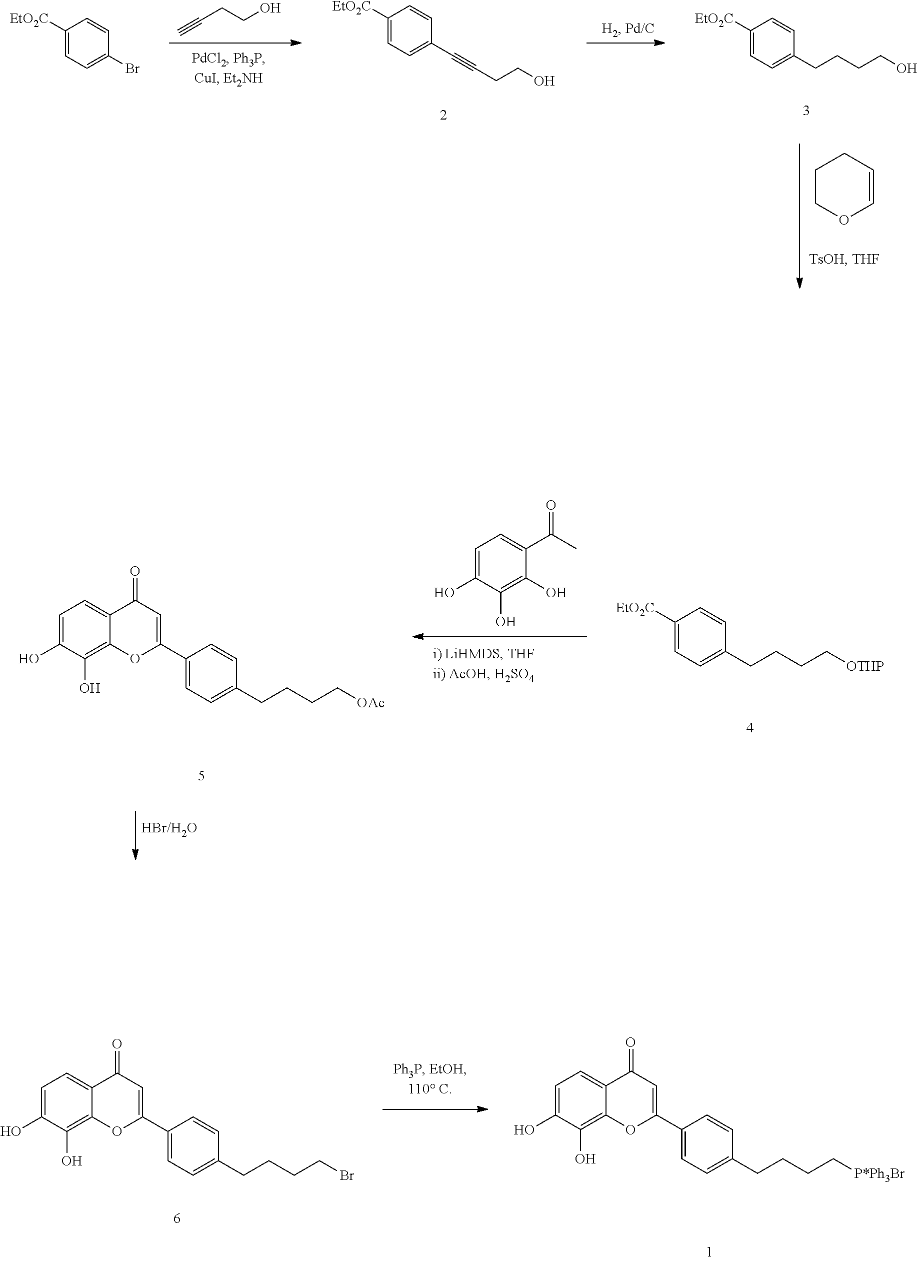

All solvents, reagents and compounds were purchased and used without further purification unless stated otherwise.

### ABBREVIATIONS

LiHMDS—Lithium bis(trimethylsilyl)amide
THF—Tetrahydrofuran
THP—Tetrahydropyran
Pd/C—Palladium on carbon (10 wt. % loading)
AcOH—Acetic acid
DCM—Dichloromethane
MeOH—Methanol
EtOH—Ethanol
Et₂NH—Diethylamine
TsOH—Toluenesulfonic acid

### Synthesis Example 1: SND118

### Ethyl 4-(4-hydroxybut-1-ynyl)benzoate (2)

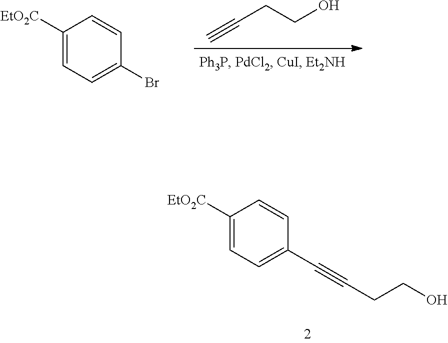

This Sonogashira coupling following a published procedure [Radeke H et al, 2007] provided 82% yield of 2.

A suspension of ethyl 4-bromobenzoate (50 g, 0.218 mol) in diethylamine (700 mL) was stirred at room temperature under nitrogen and treated with PdCl₂ (1.93 g) and triphenylphosphine (0.57 g). The mixture was de-gassed by bubbling nitrogen through for 30 min. CuI (0.42 g) and 3-butyn-1-ol (15.3 g, 0.218 mol) were added and the mixture continued at room temperature. After 20 hours more PdCl₂ (0.2 g), triphenylphosphine (0.06 g) and 3-butyn-1-ol (1.5 g) were added and continued at room temperature. After 44 hours the reaction mixture was evaporated in vacuo. Column chromatography of the residue provided ethyl 4-(4-hydroxybut-1-ynyl)benzoate (2) as a waxy solid, 39.3 g, 82-5%. 1H NMR (300 MHz, CDCl₃): δ 8.00 ppm (d, 2H), 7.48 (d, 2H), 4.39 (q, 2H), 3.84 (t, 2H), 2.72 (t, 2H), 2.88 (br s, 1H), 1.40 (t, 3H).

### Ethyl 4-(4-hydroxybutyl)benzoate (3)

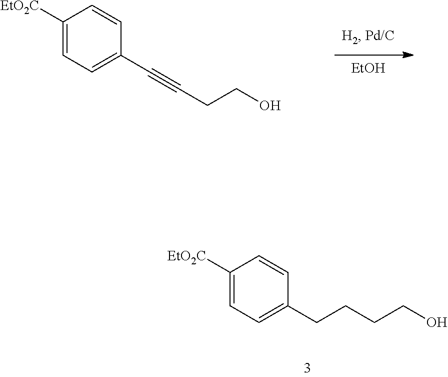 [SMILES: "EtOH" ; CCOC(=O)c1ccc(C#CCCO)cc1 ; CCOC(=O)c1ccc(CCCCO)cc1]

Hydrogenation at 40 psi pressure of hydrogen provided the saturated product (3). A solution of ethyl 4-(4-hydroxybut-1-ynyl)benzoate (41.5 g, 0.179 mol) in EtOH (300 mL) was treated with 10% Pd/C (9.51 g) and hydrogenated at 40 psi at room temperature. After 18 hours the catalyst was removed by filtration and the filtrate was evaporated in vacuo to provide ethyl 4-(4-hydroxybutyl)benzoate as an amber oil, 37.27 g, 93.8%.

1H NMR (300 MHz, CDCl₃): δ 7.98 ppm (d, 2H), 7.26 (d, 2H), 4.38 (q, 2H), 3.65 (t, 2H), 2.70 (t, 2H), 1.45-1.80 (m, 4H), 1.55 (br s, 1H), 1.40 (t, 3H).

### Ethyl 4-(4-tetrahydropyran-2-yloxybutyl)benzoate (4)

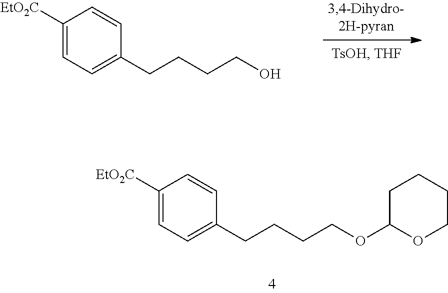 [SMILES: CCOC(=O)c1ccc(CCCCO)cc1 ; CCOC(=O)c1ccc(CCCCOC2CCCCO2)cc1]

3,4-Dihydropyran (16.4 g, 0.195 mol) in THF (50 mL) was added dropwise to a stirred solution of ethyl 4-(4-hydroxybutyl)benzoate (31.0 g, 0.139 mol) containing p-toluenesulphonic acid monohydrate (1.33 g, 6.97 mmol) in THF (320 mL) at 0° C. Warmed to room temperature for 18 hours then the reaction mixture was added to sat NaHCO₃ (700 ml) and extracted with diethyl ether (2×500 mL). The combined extracts was washed with sat. brine, dried (MgSO₄) and evaporated in vacuo. Ethyl 4-(4-tetrahydropyran-2-yloxybutyl)benzoate was obtained with good purity as an amber oil, 44.64 g, 99.8%.

1H NMR (300 MHz, CDCl₃): δ 7.87 ppm (d, 2H), 7.17 (d, 2H), 4.48 (t, 1H), 4.28 (q, 2H), 3.60-3.85 (m, 3H), 3.25-3.45 (m, 2H), 2.60 (t, 2H), 1.35-1.8 (m, 9H), 1.28 (t, 3H)

### 4-[4-(7,8-dihydroxy-4-oxo-chromen-2-yl)phenyl]butyl acetate (5)

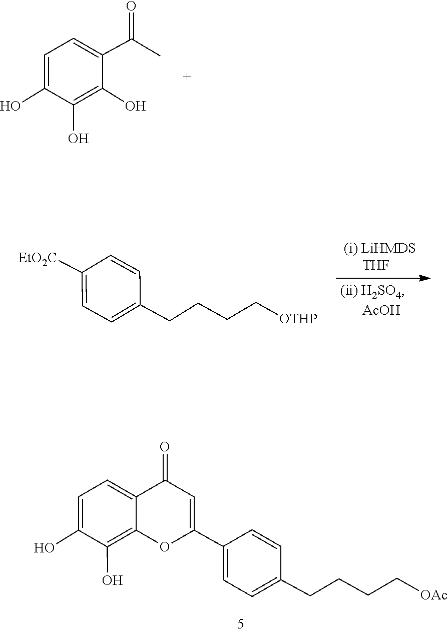

This flavone formation was carried out in two stages. The initial condensation was followed by treatment of the resulting diketone intermediate with acetic acid containing a small amount of sulphuric acid at 100° C. These conditions, in addition to effecting cyclisation to the flavone also removed the THP protection providing the acetate. 1M LiHMDS/THF solution (98.1 mL, 98.1 mmol) was added dropwise, over 30 min to a stirred solution of 2,3,4-trihydroxyacetophenone (3.3 g, 19.9 mmol) in THF (170 mL) at −70° C. Stirred 1 hour at −70° C. then warmed to −10° C. for 1 hour. Cooled back to −70° C. and a solution of ethyl 4-(4-tetrahydropyran-2-yloxybutyl)benzoate (6.3 g, 19.6 mmol) in THF (30 mL) was added dropwise over 20 min. The reaction mixture was continued at −70° C. for 1 hour then warmed to room temperature.

After 18 hours the reaction mixture was poured into ice-water (1 L) and acidified by addition of 2N HCl. Extracted with EtOAc (3×300 mL) and the combined extracts was washed with saturated brine (300 mL), dried (MgSO₄) and evaporated in vacuo. Brown oil, 11.72 g.

This oil was dissolved in glacial acetic acid (68 mL) and conc. H₂SO₄ (0.3 mL) was added. Stirred under nitrogen and heated to 100° C. for 1 hour. The dark solution was cooled, poured onto ice-water (330 mL) and extracted with EtOAc (3×150 mL). The combined extracts was washed with saturated brine (4×150 mL) and dried (MgSO₄).

Evaporated in vacuo to leave a dark oil/solid. This was triturated with dichloromethane (DCM) (30 mL) then petroleum ether (7.5 mL) was added. Stirred and cooled in an ice bath then the solid was filtered off, washed with DCM/petrol (4:1) then with petrol.

4-[4-(7,8-Dihydroxy-4-oxo-chromen-2-yl)phenyl]butyl acetate was obtained as a brown solid, 4.64 g, 64.2%.

1H NMR (300 MHz, d6-DMSO): δ 10.30 ppm (br s, 1H), 9.44 (br s, 1H), 8.07 (d, 2H), 7.40 (d, 2H), 7.40 (d, 1H), 6.95 (d, 1H), 6.83 (s, 1H), 4.02 (t, 2H), 2.70 (t, 2H), 2.00 (s, 3H), 1.50-1.70 (m, 4H).

### 2-[4-(4-bromobutyl)phenyl]-7,8-dihydroxychromen-4-one (6)

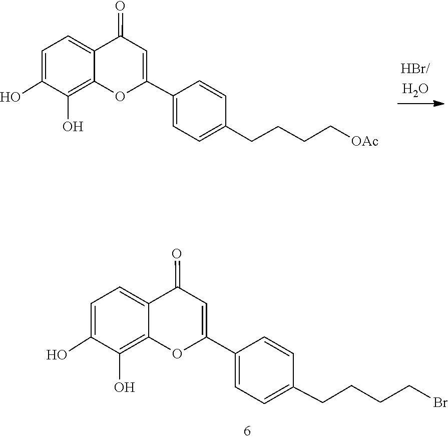 [SMILES: CC(=O)OCCCCc1ccc(-c2cc(=O)c3ccc(O)c(O)c3o2)cc1 ; O=c1cc(-c2ccc(CCCCBr)cc2)oc2c(O)c(O)ccc12]

A suspension of 4-[4-(7,8-dihydroxy-4-oxo-chromen-2-yl)phenyl]butyl acetate (4.60 g, 12.5 mmol) in 62% aqueous HBr (10.9 mL, 125 mmol) was stirred and heated to 80° C. After 5 hours the reaction mixture (light brown suspension) was cooled and treated with EtOAc (150 mL) and water (50 mL). The aqueous phase was extracted with EtOAc (2×30 mL). The combined organics was washed with water (2×100 mL), dried (MgSO₄) and evaporated in vacuo to leave a brown solid/oil, 4.58 g.

Purification by column chromatography (DCM/MeOH, 96:4) provided 2-[4-(4-bromobutyl)phenyl]-7,8-dihydroxychromen-4-one as a yellow solid, 2.139, 44%.

1H NMR (300 MHz, d6-DMSO): δ 10.30 ppm (br s, 1H), 9.44 (br s, 1H), 8.07 (d, 2H), 7.41 (d, 2H), 7.40 (d, 1H), 6.95 (d, 1H), 6.83 (s, 1H), 3.58 (t, 2H), 2.70 (t, 2H), 1.65-1.88 (m, 4H).

### 4-[4-(7,8-dihydroxy-4-oxo-chromen-2-yl)phenyl]butyltriphenyl-phosphonium bromide (i)—SND118

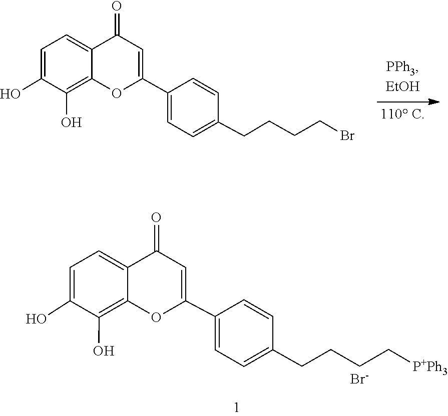

This reaction involved heating to 110° C. in a sealed vessel and was not a particularly clean reaction so required column chromatography for purification. Solvent removal from the isolated product proved difficult. Material from two separate batches was combined in ethanol solution and evaporated to a solid.

A solution of 2-[4-(4-bromobutyl)phenyl]-7,8-dihydroxychromen-4-one (1.80 g, 4.62 mmol) in EtOH (70 mL) was treated with triphenylphosphine (1.58 g, 6.01 mmol) and stirred in a sealed glass tube while heated to 110° C.

After 66 hours the solution was cooled and evaporated to a yellow foam, 3.35 g. Column chromatography (DCM/MeOH. 95:5 gradient to 90:10) provided the product at 93% purity. Further column chromatography of this material (DCM/MeOH (93:7) improved the purity to >95%, providing 4-[4-(7,8-dihydroxy-4-oxo-chromen-2-yl)phenyl]butyl-triphenylphosphonium bromide as a yellow foam, 0.75 g, 25% yield. This was combined with a second batch of similar purity prepared by the same procedure. The combined material was evaporated from ethanol to a yellow foam. After crushing to a powder and drying under vacuum 4-[4-(7,8-dihydroxy-4-oxo-chromen-2-yl)phenyl]butyl-triphenylphosphonium bromide was obtained as a yellow solid, 1.364 g, 21% yield with 97.3% HPLC purity.

1H NMR (300 MHz, d6-DMSO): δ 10.35 ppm (br s, 1H), 9.50 (br s, 1H), 8.02 (d, 2H), 7.7-7.95 (m, 15H), 7.40 (d, 1H), 7.35 (d, 2H), 6.96 (d, 1H), 6.84 (s, 1H), 3.65 (m, 2H), 2.70 (t, 2H), 1.80 (m, 2H), 1.48-1.65 (m, 2H).

Compound 1 is also referred to as compound SND118.

Other compounds can be synthesised in essentially the same way. Some further examples are provided below.

### 4-[4-(7,8-dihydroxy-4-oxo-chromen-2-yl)phenyl]butyltriphenyl-phosphonium bromide—SND118

SND118 can also be prepared by heating the bromo compound (6) with 1.4 equivalents of triphenylphosphine in acetonitrile at 110° C. in a sealed vessel.

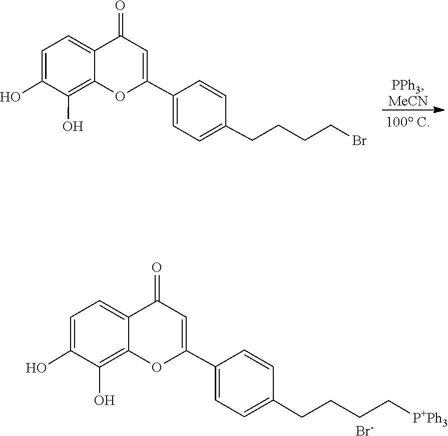

A suspension of bromo compound 6 (0.60 g, 1.54 mmol) in MeCN (30 mL) was treated with triphenylphosphine (0.57 g, 2.16 mmol) and stirred in a sealed steel vessel while heated to 110° C. After 18 h the reaction mixture consisted of a light brown solution together with some dark tar which had collected at bottom of the vessel. The reaction mixture was evaporated and column chromatographed on silica gel (97:3 DCM/MeOH) to obtain the product as an amber gum. Lyophilization from a 1:1 mixture of acetonitrile and water provided the triphenylphosphonium bromide as a yellow solid, 411 mg, 40.9%

¹H NMR (300 MHz, d₆-DMSO): δ 10.35 ppm (br s, 1H), 9.50 (br s, 1H), 8.02 (d, 2H), 7.7-7.95 (m, 15H), 7.40 (d, 1H), 7-35 (d, 2H), 6.96 (d, 1H), 6.84 (s, 1H), 3.65 (m, 2H), 2.70 (t, 2H), 1.80 (m, 2H), 1.48-1.65 (m, 2H).

### Synthesis Example 2: SND121

### 4-[4-[7-(dimethylcarbamoyloxy)-8-hydroxy-4-oxo-chromen-2-yl]phenyl]butyltriphenyl-phosphonium bromide—SND121

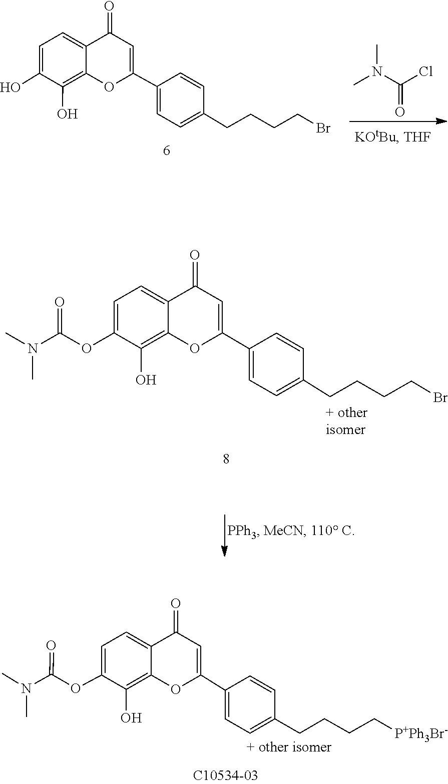

### Stage 1:

A solution of the sm (2.0 g, 5.14 mmol) in THF (50 mL) was stirred at 0° C., under nitrogen while potassium t-butoxide (1.15 g, 10.30 mmol) was added in portions. Stirred for 30 min. at ° C. then dimethylcarbamoyl chloride (0.61 g, 5.65 mmol) in THF (5 mL) was added dropwise. Warmed to room temperature and continued overnight. After 18 h, LC-MS analysis of the reaction mixture indicated a mixture containing starting material (32%), monocarbamate product (40%) and dicarbamate product (19%). The reaction mixture was treated with 1N HCl (80 mL) and EtOAc (80 mL) and the layers were separated. The aqueous phase was extracted with EtOAc (2×30 mL) and the combined organics was washed with water then extracted with 1N NaOH (2×40 mL). The combined aqueous extracts was washed with EtOAc (40 mL) then was acidified with 2N HCl and extracted with EtOAc (3×40 mL). The combined extracts was then dried (MgSO₄) and evaporated to a brown thick oil, 1.66 g. LC-MS analysis indicated this was a mixture of the starting material and mono-carbamate product which now contained only a trace amount of the dicarbamate product. Column chromatography (DCM/MeOH, 98:2) provided intermediate 8 as an off-white solid, 678 mg, 28.7%

¹H NMR (300 MHz, d₆-DMSO): (major isomer) δ 10.92 ppm (br s, 1H), 7.86 (d, 2H), 7.75 (d, 1H), 7.44 (d, 2H), 7.07 (d, 1H), 6.92 (s, 1H), 3.58 (t, 2H), 3.22 (s, 3H), 3.02 (s, 3H), 2.72 (t, 2H), 1.70-1.88 (m, 4H).

### Stage 2:

A solution of 8 (0.68 g, 1.48 mmol) in MeCN (30 mL) was treated with triphenylphosphine (0.54 g, 2.07 mmol) and stirred in a sealed steel vessel while heated to 110° C. After 18 h, the reaction mixture was evaporated in vacuo and the residue was column chromatographed (DCM/MeOH, 94:6) to obtain a foam. Lyophilization from a 1:1 MeCN/water mixture provided C10534-03 as a white solid, 303 mg, 28.4%. HPLC indicated a 94:6 mixture of the isomeric products.

¹H NMR (300 MHz, d₆-DMSO): δ 10.95 ppm (br s, 1H), 7.85-7.92 (m, 3H), 7.75-7.84 (m, 15H), 7.39 (d, 2H), 7.07 (d, 1H), 6.92 (s, 1H), 3.63 (m, 2H), 3.21 (s, 3H), 3.00 (s, 3H), 2.72 (t, 2H), 1.80 (m, 2H), 1.50-1.62 (m, 2H).

### Synthesis Example 3: SND122

### 4-[4-[7-(2,2-dimethylpropanoyloxy)-8-hydroxy-4-oxo-chromen-2-yl]phenyl]butyltriphenyl-phosphonium bromide—SND122

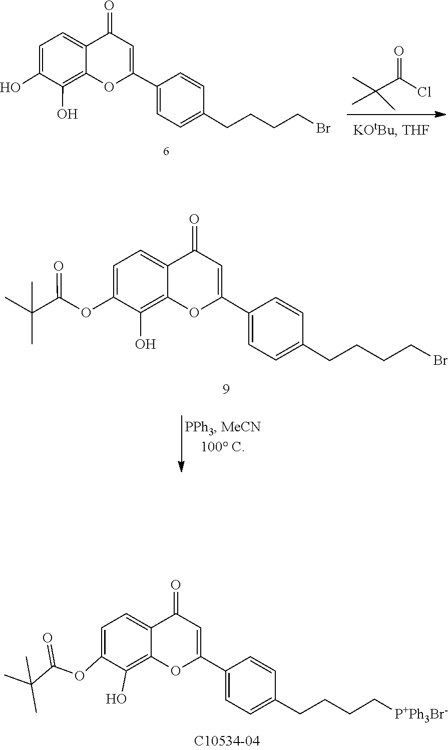

### Stage 1:

A solution of 6 (2.40 g, 6.17 mmol) in THF (100 mL) was stirred at 0° C., under nitrogen, while potassium t-butoxide (1.38 g, 12.3 mmol) was added. Stirred 30 min. then trimethylacetyl chloride (0.74 g, 6.17 mmol) in THF (20 mL) was added dropwise. Continued at 0° C. for 1 hour then allowed to warm to room temperature overnight. After 18 h the reaction mixture was treated with water (200 mL) and EtOAc (200 mL). Acidified by addition of 2N HCl and the layers were separated. The aqueous phase was extracted with EtOAc (2×100 mL) and the combined organics was washed with water (100 mL), dried (MgSO₄) and evaporated to a brown solid. This was combined with some crude product from a previous smaller batch (from 0.20 g of 6) and purified by column chromatography (DCM/MeOH, 98:2). Intermediate 9 was obtained as a beige solid, 1.35 g, 42.7%. The ¹H NMR spectrum indicated that this was a single isomer with no evidence of any of the other isomer present.

¹H NMR (300 MHz, d₆-DMSO): δ 11.08 ppm (br s, 1H), 7.87 (d, 2H), 7.80 (d, 1H), 7.41 (d, 2H), 7.10 (d, 1H), 6.90 (s, 1H), 3.58 (t, 2H), 2.70 (t, 2H), 1.70-1.88 (m, 4H), 1.42 (s, 9H).

### Stage 2:

A solution of 9 (0.65 g, 1.37 mmol) in MeCN (27 mL) was treated with triphenylphosphine (0.504 g) and stirred in a sealed vessel while heated to 110° C. After 18 h the reaction mixture was evaporated and the residue was column chromatographed (DCM/MeOH, 94:6) to obtain a gum. Lyophilization from a 1:1 MeCN/water mixture provided C10534-04 as a white solid, 351 mg, 34-7%. HPLC and NMR analysis confirmed that the product had been isolated as a single isomer.

¹H NMR (300 MHz, d₆-DMSO): δ 11.09 ppm (br s, 1H), 7.72-7.93 (m, 18H), 7.36 (d, 2H), 7.12 (d, 1H), 6.90 (s, 1H), 3.63 (m, 2H), 2.72 (t, 2H), 1.80 (m, 2H), 1-56 (m, 2H), 1.42 (s, 9H).

### Synthesis Example 4: SND123

### 4-[4-(6-oxo-[1,3]dioxolo[4,5-h]chromen-8-yl)phenyl]butyl-triphenylphosphonium bromide—SND123

SND123 was prepared by a three-step route from the acetate intermediate, 5.

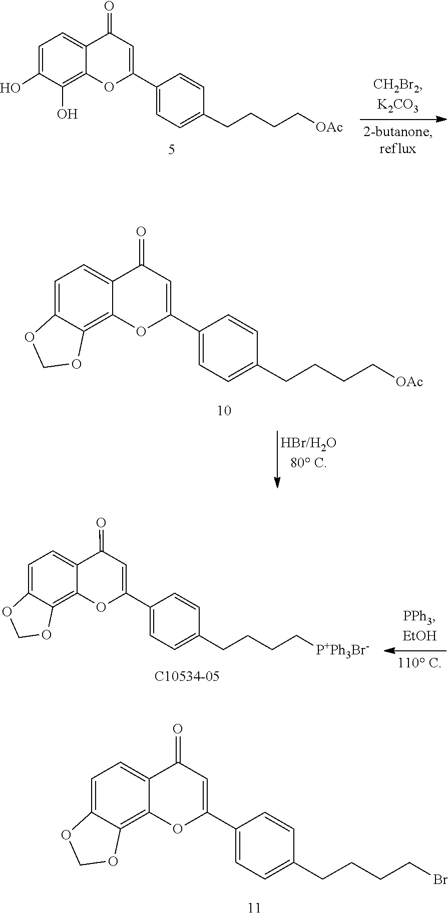

### Stage 1:

A solution of 5 (1.50 g, 4.07 mmol) in 2-butanone (20 mL) was stirred under nitrogen and treated with potassium carbonate (1.41 g, 10.2 mmol) and dibromomethane (0.57 mL, 8.14 mmol) then heated to reflux. After 4 h an additional portion of dibromomethane (2.3 mL) was added and continued at reflux overnight. After 22 h the reaction mixture was cooled and evaporated. The residual dark tar was partitioned between DCM (50 mL) and water (50 mL). The layers were separated and the aqueous phase was extracted with DCM (2×30 mL). The combined organic phases was washed with water (2×30 mL), dried (MgSO₄) and evaporated in vacuo. Column chromatography of the residual material (DCM/MeOH, 99:1) provided intermediate 10 as a beige solid, 0.74 g, 47.8%.

¹H NMR (300 MHz, CDCl₃): δ 7.87 ppm (d, 2H), 7.83 (d, 1H), 7.35 (d, 2H), 6.98 (d, 1H), 6.74 (s, 1H), 6.25 (s, 2H), 4.12 (t, 2H), 2.75 (t, 2H), 2.07 (s, 3H), 1.66-1.80 (m, 4H).

### Stage 2:

A suspension of 10 (0.70 g, 1.84 mmol) in 48% aqueous HBr (4.4 mL) was stirred under nitrogen and heated to 80° C. After 4 hours the reaction was cooled and the suspension was partitioned between EtOAc (100 mL) and water (40 mL). The aqueous phase was separated and extracted with EtOAc (2×20 mL) then the combined extracts was washed with water (3×40 mL), dried (MgSO₄) and evaporated in vacuo to a light brown oil. Column chromatography (DCM/MeOH, 99:1) provided 8-[4-(4-bromobutyl)phenyl]-[1,3]dioxolo[4,5-h]chromen-6-one (11) as a light beige solid, 0.47 g, 63-7%.

¹H NMR (300 MHz, CDCl₃): δ 7.85 ppm (d, 2H), 7.82 (d, 1H), 7.34 (d, 2H), 6.97 (d, 1H), 6.72 (s, 1H), 6.23 (s, 2H), 3.45 (t, 2H), 2.74 (t, 2H), 1.79-1.96 (m, 4H).

### Stage 3:

A suspension of 11, (0.45 g, 1.12 mmol) in EtOH (20 mL) was treated with triphenylphosphine (0.382 g, 1.46 mmol) and placed in a sealed steel vessel. Stirred and heated to 110° C. for 18 hours. The reaction mixture (brown solution) was then evaporated in vacuo and column chromatographed to provide 4-[4-(6-oxo-[1,3]dioxolo[4,5-h]chromen-8-yl)phenyl]butyl-triphenylphosphonium bromide (C10534-05) as an off-white foam. Lyophilization from 1:1 MeCN/water provided a white solid, 317 mg, 42.6% in a solvent-free state.

¹H NMR (300 MHz, d₆-DMSO): δ 7.73-7.93 ppm (m, 17H), 7.63 (d, 1H), 7.37 (d, 2H), 7.17 (d, 1H), 6.92 (s, 1H), 6.35 (s, 2H), 3.63 (m, 2H), 2.72 (t, 2H), 1.80 (m, 2H), 1-56 (m, 2H), 1.42 (s, 9H).

### Synthesis Example s: SND126A

### 4-[4-[7-(Ethylcarbamoyloxy)-8-hydroxy-4-oxo-chromen-2-yl]phenyl]butyltriphenylphosphonium bromide

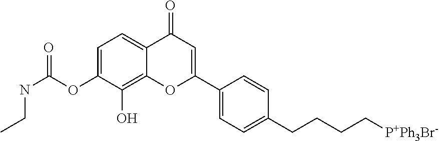

Compound 1, 4-[4-(7,8-dihydroxy-4-oxo-chromen-2-yl)phenyl]butyltriphenylphosphonium bromide, was synthesised as above. Compound SND126A was obtained according to the scheme below:

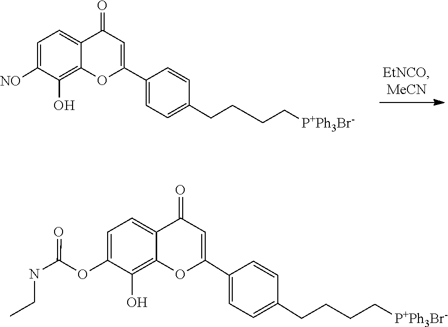

Ethyl isocyanate (0.15 mL, 1.8 mmol) was added to a solution of 4-[4-(7,8-dihydroxy-4-oxo-chromen-2-yl)phenyl]butyltriphenylphosphonium bromide (1.0 g, 1.5 mmol) in MeCN (20 mL) at 50° C. and the mixture stirred for 1 h. The solution was cooled, concentrated and the residue purified by column chromatography twice (DCM/MeOH, from 0 to 20% MeOH) to give the product as an off-white solid. A further column using DCM/DCM+10% MeOH, 0 to 100%) gave the product as an off-white solid.

1H NMR (400 MHz, d6-DMSO): δ 10.96 (1H, s, br), 8.10 (1H, t, J=5.7 Hz), 7.93-7.84 (5H, m), 7.84-7.71 (13H, m), 7.34 (2H, d, J=8.3 Hz), 7.06 (1H, d, J=8.8 Hz), 6.93 (1H, s), 3.70-3.57 (2H, m), 3.18 (2H, quint., J=6.0 Hz), 2.73 (2H, t J=7.4 Hz), 1.80 (2H, quint. J=7.2 Hz), 1.61-1.49 (2H, m), 1.15 (3H, t, J=7.2 Hz)

### Synthesis Example 6: SND127

### 4-[4-[7-(Isopropylcarbamoyloxy)-8-hydroxy-4-oxo-chromen-2-yl]phenyl]butyltriphenylphosphonium bromide

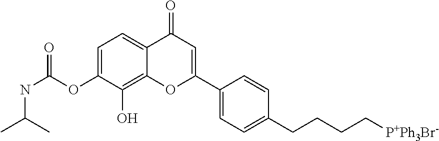

SND127 is synthesised using compound SND118 as an intermediate. The intermediate can be prepared using any synthesis described herein, including that described above in Synthesis Example 1. Alternatively, SND118 (referred to below as Intermediate 1) can be synthesised using the following general scheme:

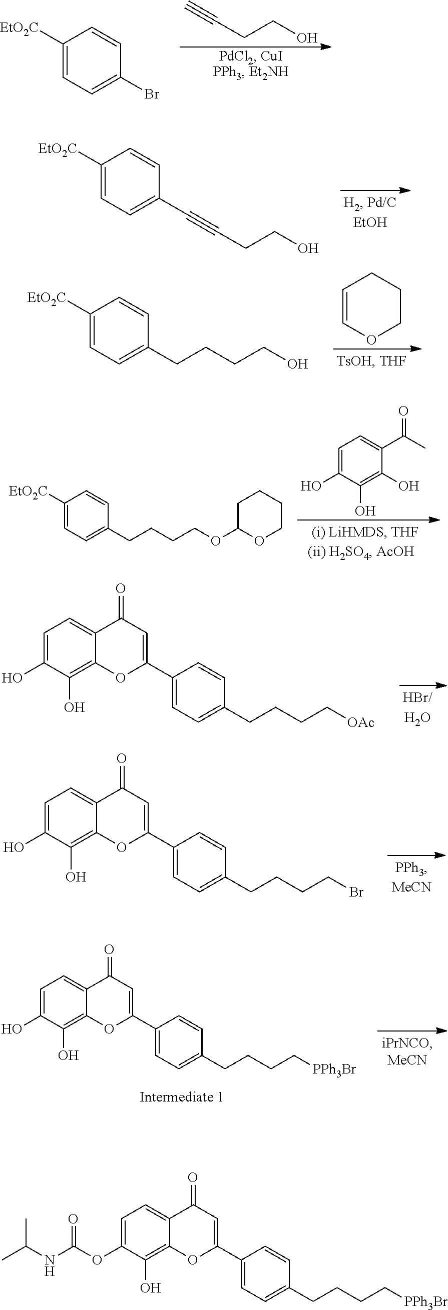

The final step is carried out using the following experimental procedure.

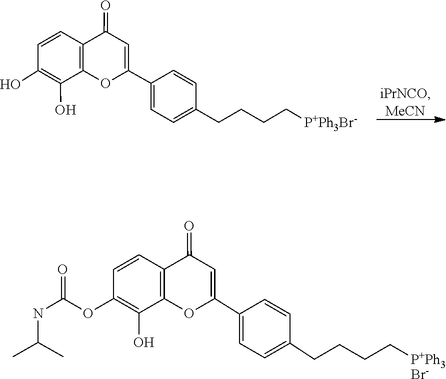

In two vials, a solution of 4-[4-(7,8-dihydroxy-4-oxo-chromen-2-yl) phenyl]butyltriphenylphosphonium bromide (0.5 g, 0.75 mmol) in MeCN (6 mL) was heated to 50° C., isopropyl isocyanate (0.09 mL, 0.9 mmol, 1.2 equivalents) and the mixture stirred for 1 h. The solution was cooled and the solids from the vials filtered off, washing through with a small amount of MeCN. The combined solids were then purified by column chromatography (DCM/MeOH, from 0 to 20%) to give an off-white solid.

This compound was analysed using SFC conditions, similar to the other compounds in this series, and showed a purity of 99%. The amount of compound obtained was 0.41 g, with a yield of 36% from intermediate 1.

¹H NMR (400 MHz, d6-DMSO): δ 10.96 (1H, s, br), 8.04 (1H, d, J=7.8 Hz), 7.92-7.86 (5H, m), 7.83-7.72 (13H, m), 7.32 (2H, d, J=8.3 Hz), 7.06 (1H, d, J=8.8 Hz), 6.93 (1H, s), 3.75-3.56 (3H, m), 2.72 (2H, t J=7.4 Hz), 1.79 (2H, quint. J=7.4 Hz), 1.62-1.49 (2H, m), 1.19 (6H, d, J=6.6 Hz)

### Synthesis Example 7: SND124

### 4-[4-[7,8-Bis(ethylcarbamoyloxy)-4-oxo-chromen-2-yl]phenyl]-butyltriphenylphosphonium bromide

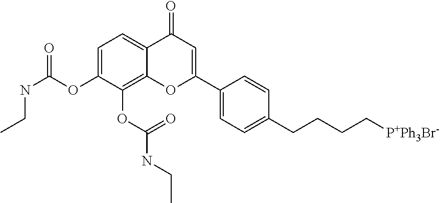

Compound SND124 was synthesised using the general scheme below:

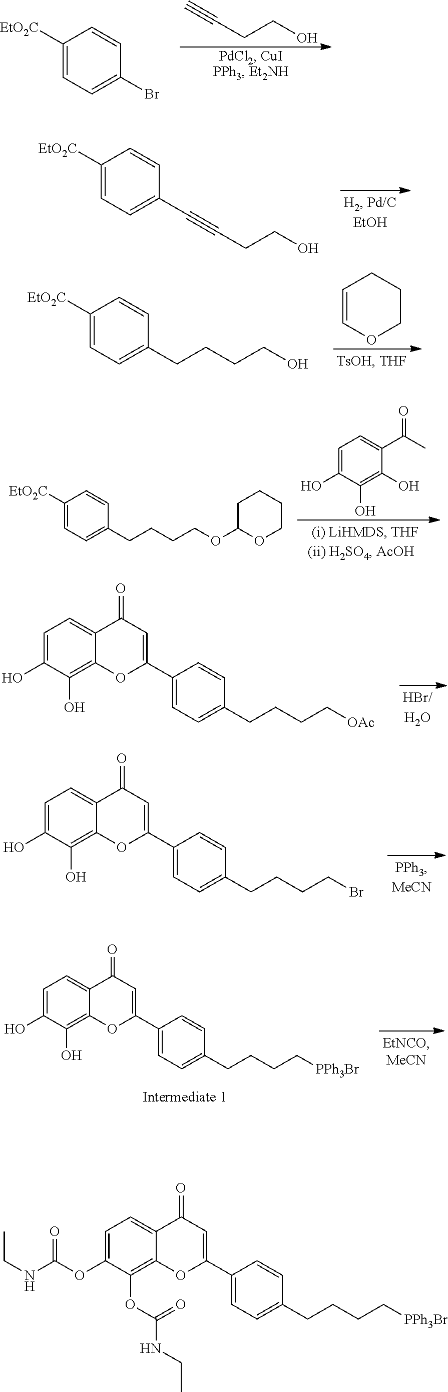

The final step is carried out using the following experimental procedure.

Ethyl isocyanate (2.4 mL, 31 mmol) was added to a solution of 4-[4-(7,8-dihydroxy-4-oxo-chromen-2-yl)phenyl]butyltriphenylphosphonium bromide (2 g, 3.1 mmol) in MeCN (30 mL) at 50° C. and the mixture stirred for 1 h. The solution was cooled, the solvent removed and the residue was purified by column chromatography (DCM/MeOH, from 0 to 20% MeOH) to give the product as an off-white solid (1.61 g, 73%).

This compound was analysed using SFC conditions, similar to the other compounds in this series, and showed a purity of 99%. The amount of compound obtained was 1.61 g, with a yield of 73% from intermediate 1.

¹H NMR (400 MHz, d6-DMSO): δ 8.33 (1H, t, J=5.5 Hz), 8.08 (1H, t, J=5.5 Hz), 7.93-7.72 (19H), 7.39-7.32 (3H, m), 7.07 (1H, s), 3.69-3-57 (2H, m), 3.23-3.07 (4H, m), 2.73 (2H, t, J=7.6 Hz), 1.85-1.75 (2H, m), 1.61-1.49 (2H, m), 1.17-1.08 (6H, m)

### Synthesis Example 8: SND 125

### 4-[4-[7,8-Bis(isopropylcarbamoyloxy)-4-oxo-chromen-2-yl]phenyl]-butyltriphenylphosphonium bromide

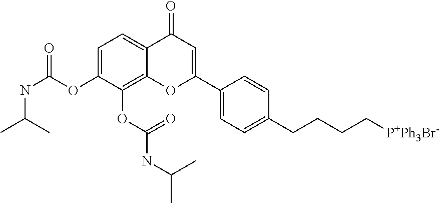

Compound SND125 was synthesised using the following scheme:

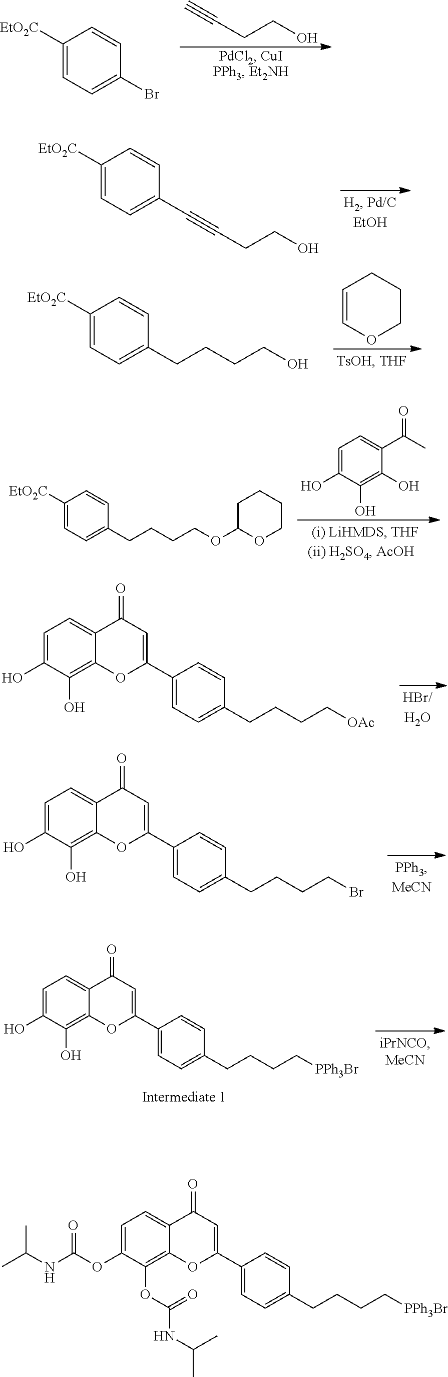

The final step was carried out using the following experimental procedure.

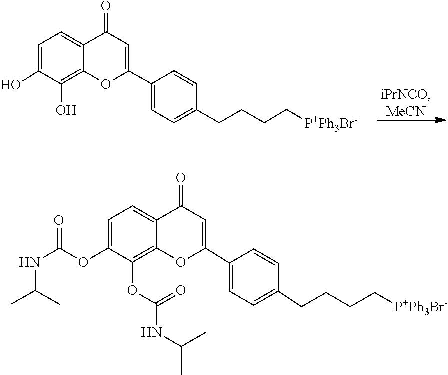

In two vials, a solution of 4-[4-(7,8-dihydroxy-4-oxo-chromen-2-yl)phenyl]butyltriphenyl-phosphonium bromide (0.25 g, 0.39 mmol) in MeCN (4 mL) was heated to 50° C. and isopropyl isocyanate (0.38 mL) was added. The mixtures were stirred for 1 h, at which point the starting material was consumed by TLC. The solutions were cooled, combined, the solvent removed and the residue was purified by column chromatography (DCM/MeOH, from 0 to 20% MeOH) three times to give the product as an off-white solid (0.1 g, 16%).

SFC conditions showed a purity of 99%. The amount obtained was 0.1 g, with an yield of 16% from intermediate 1, with the most likely reason for the poor yield being due to the repeated chromatography to reach the desired purity level.

¹H NMR (400 MHz, d6-DMSO): δ 8.25 (1H, d, J=7.7 Hz), 8.04 (1H, d, J=7.7 Hz), 7.95-7.85 (6H, m), 7.84-7.70 (12H, m), 7.35-7.30 (3H, m), 7.08 (1H, s), 3.74-3.56 (4H, m), 2.73 (2H, t, J=7.2 Hz), 1.80 (2H, quint., J=7.1 Hz), 1-56 (2H, m), 1.21-1.12 (12H, m)

### Synthesis Example 9: SND140

### (4-(4-(7-hydroxy-8-methoxy-4-oxo-4H-chromen-2-yl)phenyl)butyl)triphenylphosphonium bromide

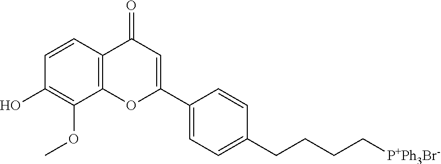

Compound SND140 was synthesised using the scheme below:

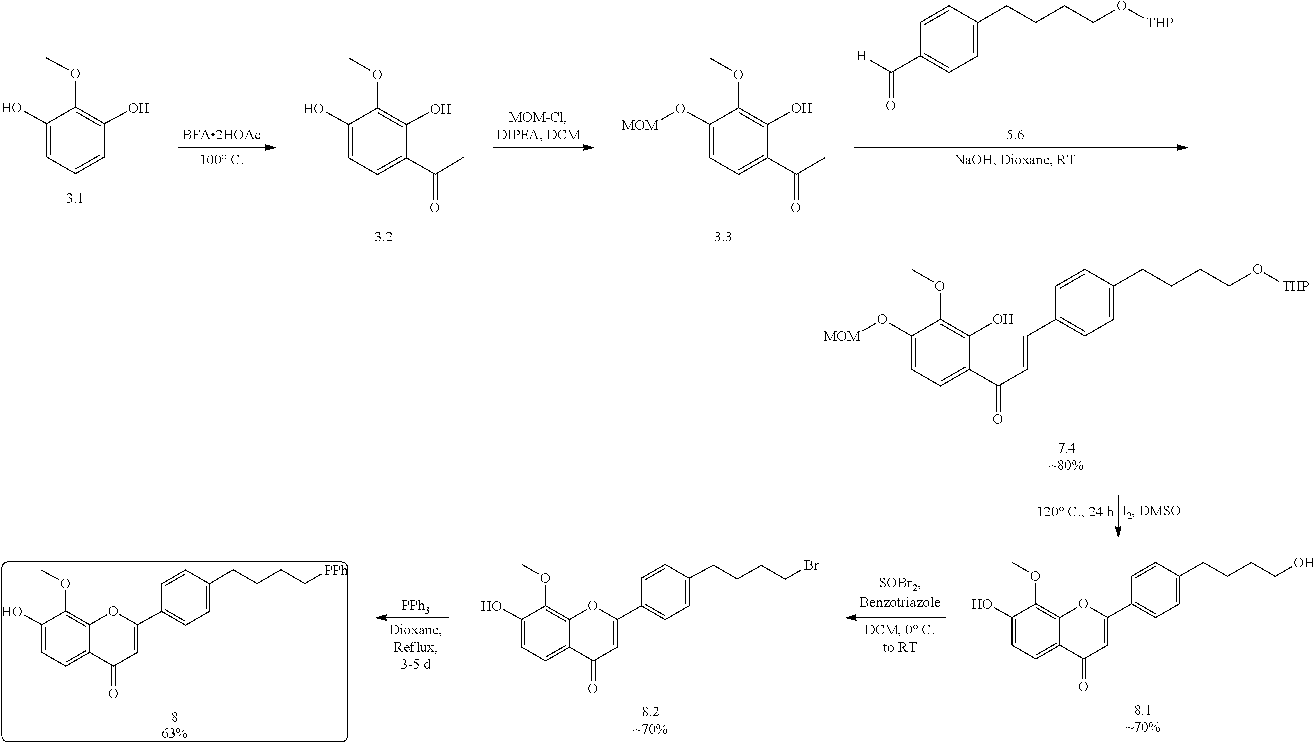

### 1-(2,4-dihydroxy-3-methoxyphenyl)ethan-1-one (3.2)

The solution of 2-methoxybenzene-1,3-diol (3.1) (0.501 g, 1.00 Eq, 3.57 mmol) in boron trifluoride-acetic acid complex (ca. 33% BF₃, 3.36 g, 2.48 mL, 5.00 Eq, 17.9 mmol) was heated to 100° C. for 180 min. The mixture was then poured into water and extracted with 20 mL DCM (3×) (Note: a leak occurred during the workup, so part of the product was lost and the yield cannot be final). The combined organic extracts were washed with brine, dried over anhydrous Na₂SO₄ and concentrated in vacuo. Resulting product 1-(2,4-dihydroxy-3-methoxyphenyl)ethan-1-one (3.2) (0.210 g, 1.15 mmol, 32.2%) was collected as dark yellow crystals.

### (E)-1-(2-hydroxy-3-methoxy-4-(methoxymethoxy)phenyl)-3-(4-(4-((tetrahydro-2H-pyran-2-yl)oxy)butyl)phenyl)prop-2-en-1-one (7.4)

To a solution of 1-(2-hydroxy-3-methoxy-4-(methoxymethoxy)phenyl)ethan-1-one (3-3) (5.00 g, 1 Eq, 22.1 mmol) and 4-(4-((tetrahydro-2H-pyran-2-yl)oxy)butyl)benzaldehyde (5.6) (6.96 g, 1.2 Eq, 26.5 mmol) in dioxane (100 mL) was added, at room temperature, aqueous sodium hydroxide (97.2 g, 97.2 mL, 50% Wt, 55 Eq, 1.22 mol). The reaction was stirred for 24 h at room temperature and controlled with LCMS until maximum conversion was reached. The solution was neutralized using citric acid, and extracted with EtOAc. The organic layers were combined, washed with brine, dried over Na₂SO₄ and concentrated in vacuo. Obtained crude material was purified by column chromatography, yielding (E)-1-(2-hydroxy-3-methoxy-4-(methoxymethoxy)phenyl)-3-(4-(4-((tetrahydro-2H-pyran-2-yl)oxy)butyl)phenyl)prop-2-en-1-one (7.4) (8.67 g, 16 mmol, 72%, 86% Purity) as a dark-orange thick oil.

### 7-hydroxy-2-(4-(4-hydroxybutyl)phenyl)-8-methoxy-4H-chromen-4-one (8.1)

A stirred solution of (E)-1-(2-hydroxy-3-methoxy-4-(methoxymethoxy)phenyl)-3-(4-(4-((tetrahydro-2H-pyran-2-yl)oxy)butyl)phenyl)prop-2-en-1-one (7.4) (6.000 g, 1 Eq, 12.75 mmol) and iodine (323.6 mg, 0.1 Eq, 1.275 mmol) in DMSO (100 mL) was heated to 120° C. for 48 hours. Upon LCMS-confirmed completion, the mixture was cooled and poured into cold water. The mixture was extracted with ethyl acetate (4×200 mL). The combined organic phase was washed with saturated sodium thiosulfate, water and brine successively. Then the organic layer was dried with anhydrous Na₂SO₄ and concentrated in vacuo. 7-hydroxy-2-(4-(4-hydroxybutyl)phenyl)-8-methoxy-4H-chromen-4-one (8.1) (3.92 g, 8.8 mmol, 69%, 76% Purity) was obtained as a viscous dark orange oil, which solidifies upon applying friction.

### 2-(4-(4-bromobutyl)phenyl)-7-hydroxy-8-methoxy-4H-chromen-4-one (8.2)

To a solution of the 7-hydroxy-2-(4-(4-hydroxybutyl)phenyl)-8-methoxy-4H-chromen-4-one (8.1) (1.50 g, 1.0 Eq, 4.41 mmol) in DCM at 0° C. was added 1H-benzo[d][1,2,3]triazole (682 mg, 1.30 Eq, 5.73 mmol) and a drop of DMF (32.2 mg, 0.1 Eq, 441 μmol), followed by sulfurous dibromide (1.19 g, 444 μL, 1.30 Eq, 5.73 mmol). The mixture was allowed to warm to room temperature and then the reaction progress was monitored by LCMS. Upon completion, the mixture was quenched with saturated aqueous NaHCO₃, and extracted with DCM (3×100 mL). The combined organic layers were washed with brine, dried (Na₂SO₄), and concentrated in vacuo. The resulting oil was purified by column chromatography (SiO₂, 0-20% MeOH/DCM) to provide 2-(4-(4-bromobutyl)phenyl)-7-hydroxy-8-methoxy-4H-chromen-4-one (8.2) (0.938 g, 2.33 mmol, 52.8%) as a light-brown solid.

### (4-(4-(7-hydroxy-8-methoxy-4-oxo-4H-chromen-2-yl)phenyl)butyl)triphenylphosphonium bromide (8)

To a solution of 2-(4-(4-bromobutyl)phenyl)-7-hydroxy-8-methoxy-4H-chromen-4-one (8.2) (0.352 g, 1.0 Eq, 873 μmol) and sodium iodide (19.6 mg, 0.15 Eq, 131 μmol) in dioxane (15 mL) was added triphenylphosphine (6.87 g, 30 Eq, 26.2 mmol) and the resulting mixture was heated to reflux (105° C.). Reaction progress was controlled by TLC (DMC/MeOH—9:1). Upon completion, which took 18 hours, the solvent was removed in vacuo and the residue was combined with a previous batch (#53, 250 mg) and triturated with water/toluene/acetone. Part of the solid remained undissolved in DCM and appeared to be the product (batch A, 425 mg, yellow powder, 97% purity). The DCM filtrate was purified by column chromatography (SiO₂, 0-20% MeOH/DCM). yielding (4-(4-(7-hydroxy-8-methoxy-4-oxo-4H-chromen-2-yl)phenyl)butyl)triphenylphosphonium bromide (8), (batch B, 115 mg, brown-yellow powder, 97% purity). Combined, 52% yield.

### Synthesis Example 10: SND176

SND176 was synthesized using the following route of synthesis.

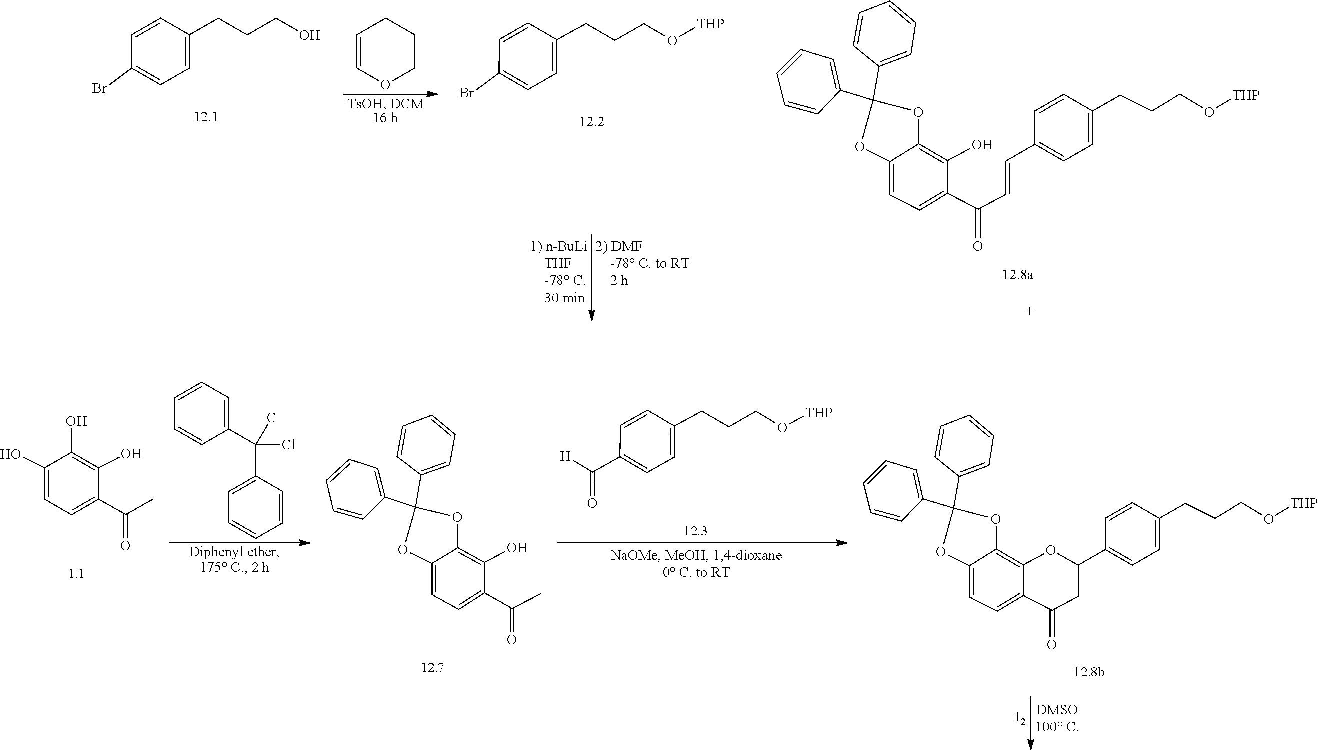

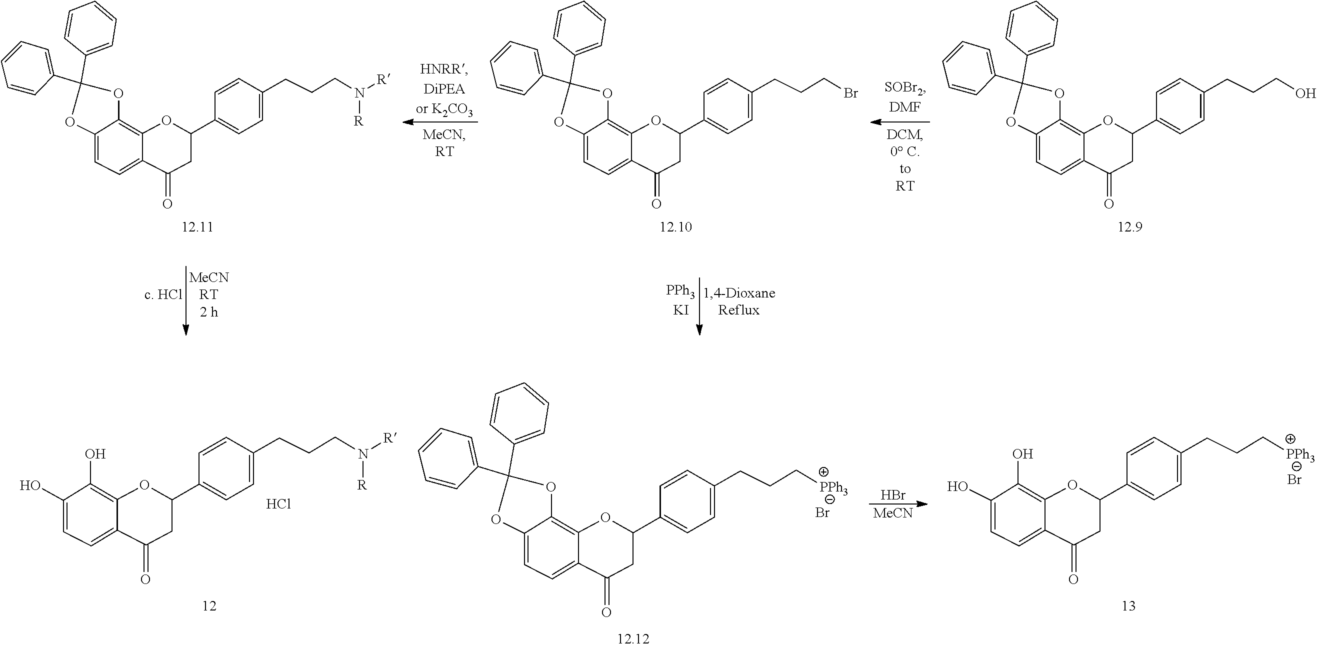

### 2-(3-(4-Bromophenyl)propoxy)tetrahydro-2H-pyran (12.2)

A solution of 3-(4-bromophenyl)propan-1-ol (12.1) (24.97 g, 1 Eq, 116.1 mmol) in dichloromethane (250 mL) was cooled under gentle nitrogen flow to 0° C. in a 500 ml round-bottom flask. p-Toluenesulfonic acid monohydrate (2.21 g, 0.111 Eq, 12.8 mmol) was then added portion-wise. 3,4-Dihydro-2H-pyran (19.35 g, 1.981 Eq, 230.0 mmol) was added drop-wise from a dropping funnel within 30 min before the mixture was allowed to warm to room-temperature. The solution turned eventually to black. The reaction mixture was stirred at room temperature for 16 hours before it was concentrated. The resultant black oil was purified by flash-chromatography using ethyl acetate/heptanes to yield 2-(3-(4-bromophenyl)propoxy)tetrahydro-2H-pyran (12.2) (28.8 g, 96.3 mmol, 83%, 100% purity) as a transparent oil.

### 4-(3-((Tetrahydro-2H-pyran-2-yl)oxy)propyl)benzaldehyde (12.3)

2-(3-(4-Bromophenyl)propoxy)tetrahydro-2H-pyran (12.2, 27.67 g, 1 Eq, 92.48 mmol) and THF (310 mL) were transferred under nitrogen flow to a flame-dried 500 ml three-neck round-bottom flask. The solution was cooled under gentle nitrogen flow to −75° C., before n-butyllithium (6.49 g, 40.5 mL, 2.5 molar, 1.09 Eq, 101 mmol) in hexanes was added portion-wise within 20 min. After 30 min of stirring, dry DMF was added portion-wise within 25 min and the reaction mixture was stirred for another 5 min before the cooling bath was removed. The reaction mixture was then stirred at 20° C. for 2 hour, before the reaction mixture was quenched with 100 ml of water and diluted with 900 ml of water. Resulting suspension was extracted with 3×750 ml of EtOAc. The organic fractions were combined, dried with sodium sulfate, filtered and concentrated to give the crude product as a yellow oil. The crude product was purified by flash-chromatography using ethyl acetate/heptanes to yield 4-(3-((tetrahydro-2H-pyran-2-yl)oxy)propyl)benzaldehyde (12.3) (18.7 g, 75 mmol, 81%, 99% purity) as a colorless oil.

### 1-(4-Hydroxy-2,2-diphenylbenzo[d][1,3]dioxol-5-yl)ethan-1-one (12.7)

1-(2,3,4-Trihydroxyphenyl)ethan-1-one (10.86 g, 1 Eq, 64.59 mmol), dichlorodiphenylmethane (15.29 g, 12.38 mL, 1.00 Eq, 64.48 mmol) and diphenyl ether (85 mL) were transferred under nitrogen flow to a 250 ml three-neck flask. The reaction mixture was heated at 175° C. for 30 min. The reaction mixture was allowed to cool to room temperature before it was poured to 900 ml of heptane. After a couple of minutes, precipitate started to form. This was filtered and washed with heptane. The dark precipitate on the filter was dissolved in DCM, 25 mL of EtOAc and 25 mL of heptane was added. This mixture was then concentrated until extensive precipitate formed. This was filtered, washed with 4×25 mL of EtOAc:heptane 1:1 mixture and purified by normal phase flash-chromatography using EtOAc:heptane as the eluent.

The filtrate of the first filtration was concentrated, cooled to 4° C. for 20 h, filtered and washed with heptane This was combined with the material recovered from flash-chromatography to yield 1-(4-hydroxy-2,2-diphenylbenzo[d][1,3]dioxol-5-yl)ethan-1-one (12.7) (15.62 g, 47.0 mmol, 73%, 100% purity) as a white solid.

### (E)-1-(4-Hydroxy-2,2-diphenylbenzo[d][1,3]dioxol-5-yl)-3-(4-(3-((tetrahydro-2H-pyran-2-yl)oxy)propyl)phenyl)prop-2-en-1-one (12.8a) and 2,2-diphenyl-8-(4-(3-((tetrahydro-2H-pyran-2-yl)oxy)propyl)phenyl)-7,8-dihydro-6H-[1,3]dioxolo[4,5-h]chromen-6-one (12.8b)

Sodium methoxide (379 g, 130 mL, 5.4 molar, 35.7 Eq, 702 mmol) in MeOH was added portion-wise under nitrogen flow to an ice/NaCl cooled suspension of 1-(4-hydroxy-2,2-diphenylbenzo[d][1,3]dioxol-5-yl)ethan-1-one (6.5287 g, 1 Eq, 19.643 mmol) and 4-(3-((tetrahydro-2H-pyran-2-yl)oxy)propyl)benzaldehyde (5.037 g, 1.033 Eq, 20.28 mmol) in 1,4-dioxane (70 mL) at 0° C. The mixture was allowed slowly to warm to room temperature and it was stirred for 15 h under nitrogen atmosphere. The reaction mixture was then poured to 500 ml of ice-cold brine. The resultant suspension was extracted with 3×100 ml of EtOAc. Organic fractions were combined, dried with sodium sulfate, filtered and evaporated to dryness, yielding 14.27 g of dark orange oil. The crude product was suspended in DCM and purified twice by normal phase flash-chromatography using DCM:MeOH as the eluent, to yield (E)-1-(4-hydroxy-2,2-diphenylbenzo[d][1,3]dioxol-5-yl)-3-(4-(3-((tetrahydro-2H-pyran-2-yl)oxy)propyl)phenyl)prop-2-en-1-one (12.8a) (5.16 g, 9.17 mmol, 46.7%, 100% purity) as an orange foam and 2,2-diphenyl-8-(4-(3-((tetrahydro-2H-pyran-2-yl)oxy)propyl)phenyl)-7,8-dihydro-6H-[1,3]dioxolo[4,5-h]chromen-6-one (12.8b) (4.825 g, 7.7 mmol, 39%, 90% purity) a yellow foam.

### 2,2-Diphenyl-8-(4-(3-((tetrahydro-2H-pyran-2-yl)oxy)propyl)phenyl)-7,8-dihydro-6H-[1,3]dioxolo[4,5-h]chromen-6-one (12.9)

A solution of 2,2-diphenyl-8-(4-(3-((tetrahydro-2H-pyran-2-yl)oxy)propyl)phenyl)-7,8-dihydro-6H-[1,3]dioxolo[4,5-h]chromen-6-one (12.8b) (4.825 g, 1 Eq, 8.575 mmol) and diiodine (223 mg, 0.102 Eq, 879 μmol) in DMSO (60 mL) was heated at 120° C. for 17 h. Reaction mixture was then allowed to cool to room temperature before it was poured to 600 ml of 1% sodium sulfite solution. Brown precipitate formed. The organic layer was extracted with 3×250 ml of EtOAc. Brine was added to speed up the separation of layers. Organic layers were combined and washed with 200 ml of brine, which in turn was extracted with 100 ml of EtOAc. Organic layers were combined, dried with sodium sulfate, filtered and evaporated to dryness to yield 3.93 g of dark oil. Crude product was suspended in DCM and purified by normal phase flash-chromatography. 8-(4-(3-Hydroxypropyl)phenyl)-2,2-diphenyl-6H-[1,3]dioxolo[4,5-h]chromen-6-one (12.9) (2.151 g, 4.51 mmol, 52.6%, 100% purity) was obtained as a pale yellow solid.

### 8-(4-(3-Bromopropyl)phenyl)-2,2-diphenyl-6H-[1,3]dioxolo[4,5-h]chromen-6-one (12.10)

8-(4-(3-Hydroxypropyl)phenyl)-2,2-diphenyl-6H-[1,3]dioxolo[4,5-h]chromen-6-one (12.9, 2.15 g, 1 Eq, 4.51 mmol) was dissolved in dry DCM (36 mL) and was cooled to 0° C. under nitrogen atmosphere. N,N-dimethylformamide (0.9 g, 0.9 mL, 3 Eq, 0.01 mol) was then added under nitrogen flow, followed by sulfurous dibromide (1.2 g, 0.45 mL, 1.3 Eq, 5.8 mmol). After a few minutes, the cooling bath was removed and the orange solution was stirred at 20° C. Reaction was followed by LC-MS. After 105 min, the reaction mixture was cooled with ice-bath and 50 ml of sat. NaHCO₃ was added. The mixture was then extracted with 3×100 ml of DCM, until last fraction had very little UV-activity. Organic layers were combined, washed with 150 ml of brine, which in turn was extracted with 2×50 ml of DCM and dried with sodium sulfate. The solution was filtered, evaporated to dryness and purified by normal phase flash-chromatography, using EtOAc:heptane as the eluent. 8-(4-(3-Bromopropyl)phenyl)-2,2-diphenyl-6H-[1,3]dioxolo[4,5-h]chromen-6-one (2.014 g, 3.73 mmol, 82.8%, 100% purity) was obtained as a white solid.

### (3-(4-(6-Oxo-2,2-diphenyl-6H-[1,3]dioxolo[4,5-h]chromen-8-yl)phenyl)propyl)triphenylphosphonium bromide (12.12)

Triphenylphosphine (305 mg, 6.09 Eq, 1.16 mmol) was added to a solution of 8-(4-(3-bromopropyl)phenyl)-2,2-diphenyl-6H-[1,3]dioxolo[4,5-h]chromen-6-one (12.10) (0.103 g, 1.00 Eq, 191 μmol) and sodium iodide (4.29 mg, 0.15 Eq, 28.6 μmol) in dioxane (3 mL) and the resulting mixture was heated to reflux. Reaction progress was controlled by TLC and LC-MS. The mixture was heated for 18 h, before it was allowed to cool to room temperature, filtered and washed with 3×5 mL of toluene. (3-(4-(6-Oxo-2,2-diphenyl-6H-[1,3]dioxolo[4,5-h]chromen-8-yl)phenyl)propyl)triphenylphosphonium bromide (132 mg, 165 μmol, 86.2%) was obtained as a white powder.

### (3-(4-(7,8-Dihydroxy-4-oxo-4H-chromen-2-yl)phenyl)propyl)triphenylphosphonium bromide (13)

(3-(4-(6-Oxo-2,2-diphenyl-6H-[1,3]dioxolo[4,5-h]chromen-8-yl)phenyl)propyl)triphenylphosphonium bromide (12.12) (122 mg, 1 Eq, 152 μmol) was suspended in MeCN (0.5 mL) and deprotected with c. HBr (641 mg, 433 μL, 48% Wt, Eq, 3.80 mmol). After concentration, filtration, washing and drying, (3-(4-(7,8-dihydroxy-4-oxo-4H-chromen-2-yl)phenyl)propyl)triphenylphosphonium bromide (79 mg, 0.12 mmol, 78%, 96% purity) was obtained as an orange powder.

### Examples—Biological Studies

### Experimental Methodology

Antitumor activity of the compounds and doxorubicin as a positive control was assessed by using the CellTiter-Blue Cell Viability Assay (Promega, #G8082) or CellTiter-Glow® Luminescent Cell Viability assay (Promega #G7572) according to the manufacturer's instructions. The compounds were tested at 5 or 6 concentrations in half-log increments (highest concentration 30 μM or 100 μM) in duplicate or triplicate well conditions.

Tumor cells were grown at 37° C. in a humidified atmosphere with 5% CO₂ in RPMI 1640 or DMEM medium, supplemented with 10% (v/v) fetal calf serum and 50 μg/ml gentamicin for up to 20 passages, and were passaged once or twice weekly. Cells were harvested using TrypLE or PBS buffer containing 1 mM EDTA, and the percentage of viable cells is determined using a CASY Model TI cell counter (OMNI Life Science). Cells were harvested from exponential phase cultures, counted and plated in 96 well flat-bottom microtiter plates at a cell density depending on the cell line's growth rate (4,000-20,000 cells/well depending on the cell line's growth rate, up to 60,000 for hematological cancer cell lines) in RPMI 1640 or DMEM medium supplemented with 10% (v/v) fetal calf serum and 50 μg/ml gentamicin (140 μl/well). Cultures were incubated at 37° C. and 5% CO₂ in a humidified atmosphere. After 24 h, 10 μl of test compounds or control medium were added, and left on the cells for another 72 h. Compounds were serially diluted in DMSO, transferred in cell culture medium, and added to the assay plates. The DMSO concentration was kept constant at <0.3% v/v across the assay plate. Viability of cells was quantified by the CellTiter-Blue® cell viability assay (Promega G8081) or CellTiter-Glow® Luminescent Cell Viability assay (Promega #G7572). Fluorescence (FU) was measured by using the EnSpire® multimode plate reader (Perkin Elmer) (excitation X=570 nm, emission λ=600 nm). Luminescence was measured with a microplate luminometer (Promega or PerkinElmer).

Sigmoidal concentration-response curves were fitted to the data points (test-versus-control, T/C values) obtained for each tumor model using 4 parameter non-linear curve fit (Charles River DRS Datawarehouse Software) or with GraphPad prism 5.02 software. IC₅₀ values are reported as absolute IC₅₀ values, being the concentration of test compound at the intersection of the concentration-response curves with T/C=50% Cell lines tested are presented in Table 1.

TABLE 1
Tumour cell lines type and designation

| Tumour model | Cell line |
|---|---|
| Brain | U-87 |
| | SF-268 |
| | U-251 |
| | IMR-5-75 |
| | SK-N-SH |
| | Kelly |
| | SK-N-AS |
| | SH-SY-5Y |
| | CHP-134 |
| | U-118 |
| Breast | MCF-7 |
| | MDA-MB-468 |
| | BT-747 |
| | MDA-MB-436 |
| | MDA-MB-231 |
| | HCC1806 |
| | ZR-75-1 |
| | T47D |
| Colon | HC-T116 |
| | HT-29 |
| | HCT-15 |
| | LoVo |
| | KM-12 |
| Leukemia | K-562 |
| | HL-60 |
| Lung (NSCLC) | A-549 |
| | H-1299 |
| | Calu-6 |
| | NCI-H460 |
| Lung (SCLC) | H69AR |
| | NCI-H69 |
| | DMS-114 |
| Lymphoma | U937 |
| | Farage |
| Ovarian | SK-OV-3 |
| | OVCAR-3 |
| | 899 |
| Pancreatic | Mia-Pa-Ca-2 |
| | BxPC-3 |
| | Panc-1 |
| Prostate | PC-3 |
| | LNCaP |
| | 22Rv1 |
| Renal | 486L |
| Skin (Melanoma) | A375 |
| | SK-Mel-28 |
| | SK-Mel-5 |
| | A2058 |
| | MeWo |

### Antitumor Activity Against a Panel of Patient-Derived Xenografts (PDX)

PDX-derived cell cultures were obtained from tumors explanted from mice and isolated by mechanical and enzymatic dissociation. Assays were performed on cells from frozen stocks at least 2 weeks after thawing and maintained in culture at 37° C. in a humidified atmosphere with 5% CO₂ in complete growth medium supplemented with 8 to 16% fetal bovine serum, 1% Penicillin-Streptomycin (10,000 U/mL), 2 mM L-Glutamine+/−Insulin-Transferrin-Selenium 1× and Albumax II (10 to 40 μM depending on cell type). Cells were harvested and seeded in 96-wells plates at a density of 1.25 to 5×10³ cells/well for cytotoxicity assays. Cells were incubated 48 h at 37° C. prior to addition of test molecules and vehicle (DMSO, 0.1%) at desired final concentrations. Cell viability was assessed before drugs' addition (To) and 5 days after test molecules addition by measuring ATP cell content using CellTiter-Glo® Luminescent Cell Viability Assay (Promega) according to the manufacturer's instructions. Luciferase activity was measured on a luminometer (PerkinElmer® EnVision™). Each concentration of compounds was tested in triplicate.

Viability was calculated as a percentage of ATP value compared to vehicle treated controls.

For PDX primary cell cultures, the tumour tissue was washed with PBS containing antibiotic-antimycotic and non-tumour tissue and necrotic tumour tissues were separated. The tumour tissue was transferred to a new dish and cut into 1-2 mm³ fragments, resuspended in RPMI-1640 medium and centrifuged at 1,200 rpm for 6 min at room temperature. The pelleted material was resuspended with 15 mL of Tumour Cell Digestion Solution and incubated at 37° C. for 1 hour with agitation. Following further addition of media, centrifugation and passage through a 70 mm cell strainer, the homogenous cell mixture was layered onto 15 mL of Ficoll-Paque PLUS in a 50 mL conical tube and centrifuged for 15 min at 1,600 rpm. The interface cells were collected, washed with media, separated by centrifugation at 1,200 rpm. The cell pellet was resuspended in serum free media supplemented with growth factors. 10,000 cells/wells were plated in a 96 well plate and incubated at 37° C., 5% CO₂, 95% air and 100% relative humidity overnight. The cytotoxicity assay was conducted as above. IC50 values represent absolute IC50.

PDX tested are presented in Table 2.

TABLE 2
PDX origin and designation

| Tumour organ | Model ID |
|---|---|
| Bile duct | CH-17-0091 |
| | CH-17-0098 |
| Brain | GBM14-CHA |
| | ODA14-RAV |
| Breast | HBCx-2 |
| | HBCx-3 |
| | HBCx-6 |
| | BR-05-0300 |
| | BR-05-0014E |
| Colon | TC71 |
| | CO-04-0722 |
| | CO-04-0701 |
| | CO-04-0700 |
| Endometrium | END4-HIR |
| | EN11-01-01 |
| Esophagus | ES-06-0002 |
| | ES-06-0122 |
| Head and neck | HN-13-0020 |
| Kidney | Ki-12-0062 |
| Liver | HB-214-FOI |
| Lung | IC20-DAN |
| | SC6 |
| | LU-01-0027 |
| | LU-01-0010 |
| | LU-01-0604 |
| | LU-01-0025 |
| Lymphoma | LY-24-0304 |
| Ovary | OVA2-BUR |
| Pancreas | PC-07-0045 |
| | PC-07-0059 |
| Prostate | HID28 |
| Skin | MCM002-FJ |
| | ME-21-0028 |
| Stomach | ST-02-0007 |
| | ST-02-0173 |
| | ST-02-0012 |
| | ST-02-0322 |

### Inhibition of Kinase Activity

Selected compounds were screened for kinase inhibition using the KINOMEscan™ assay (Eurofins) which is based on a competition binding assay that quantitatively measures the ability of a compound to compete with an immobilized, active-site directed ligand. The assay was performed by combining three components: DNA-tagged kinase; immobilized ligand; and the test compound. The ability of the test compound to compete with the immobilized ligand was measured via quantitative PCR of the DNA tag.

Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then resuspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

Compounds were initially tested at a concentration of 10 mM against a panel of 30 kinases and results for primary screen binding interactions were reported as ‘% Ctrl’

% Ctrl Calculation=(test compound signal−positive control signal/negative control signal−positive control signal)×100, where negative control=DMSO (100% Ctrl) positive control=control compound (0% Ctrl). Test compounds with % Ctrl between 0 and 10 were selected for Kd determination.

Binding constants (Kds) were calculated with a standard dose-response curve using the Hill equation: Response=Background+[Signal−Background/i+(Kd^{Hill Slope}/Dose^{Hill Slope})]. The Hill Slope was set to −1. Curves were fitted using a non-linear least square fit with the Levenberg-Marquardt algorithm.

### In Vivo Activity Against Human Tumour Xenografts

In vivo activity against a number of CDX were evaluated in various nude or SCID mice. Tumour cells were inoculated subcutaneous (s.c) into the mice flank. Mice were randomized into groups when tumours were around 100-200 mm³. A vehicle control group (2.5-4% DMSO, 5% EtOH, 20% PEG200 in saline) was part of each experiment. Treatment was administered intraperitoneally (i.p.) 3 times a week or daily (qd).

Body weight was measured before each treatment and treatments of individual mice was paused, when the body weight loss was >15%. Tumor volumes were measured 2×/week. Mice were individually sacrificed, when the tumor volume reached the volume specified in the laboratory internal SOP.

The tumour inhibition growth was assessed as the optimal T/C, where T represents the median tumour volume of the treated group and C represents the median tumour volume of the control (vehicle) group. Statistical analysis was performed using two way RM ANOVA with p values <0.05% considered statistically significant.

All procedures related to animal handling, care and the treatment in the study were performed according to the guidelines of the Association for Assessment and Accreditation of Laboratory Animal Care (AAALAC).

### Example 1. Activity Against Bile Duct Patient-Derived Xenografts (PDX)

SND123, SND124, SND126A, SND127 and SND140 inhibited bile duct PDX growth with IC50 below 20 μM as presented in Table 3

TABLE 3
IC50 values against bile duct carcinomas

| PDX/IC50 (μM) | CH-17-0091 | CH-17-0098 |
|---|---|---|
| SND123 | 3 | 0.72 |
| SND124 | 5.4 | 6.10 |
| SND126A | 7.2 | 8.50 |
| SND140 | >50 | 4.98 |

### Example 2. Activity Against Brain Carcinoma Cell Lines

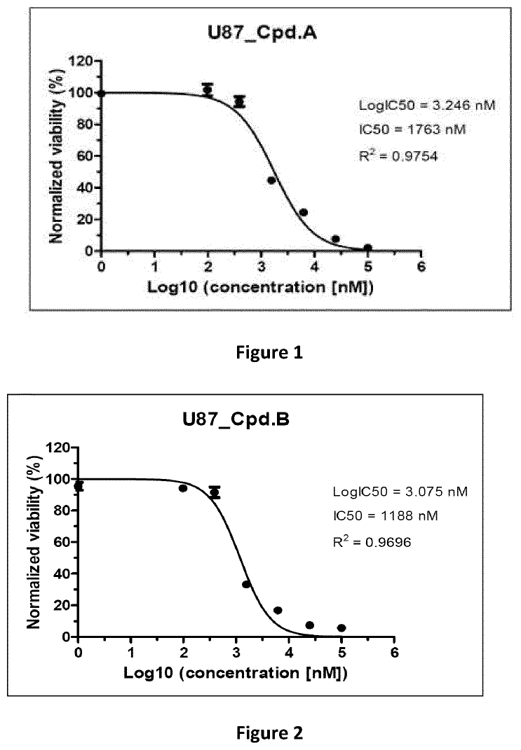

SND118, SND123, SND124, SND126A, SND127 and SND140, inhibited brain cancer cell growth with IC50s below 20 μM, as presented in Tables 4A-4D and FIG. 1.

TABLE 4A
IC50 values against brain carcinoma cell lines

| Cell Line/IC50 (μM) | U-87 |
|---|---|
| SND118 | 1.76 |
| SND123 | 0.87 |
| SND124 | 1.18 |
| SND127 | 3.8 |
| SND140 | 5.4 |

TABLE 4B
IC50 values against brain carcinoma cell lines

| Cell Line/IC50 (μM) | SF-268 |
|---|---|
| SND118 | 15.6 |
| SND123 | 3.15 |
| SND126A | 10.67 |
| SND127 | 11.9 |
| SND140 | 9.5 |

TABLE 4C
IC50 values against brain carcinoma cell lines

| Cell Line/IC50 (μM) | U-251 |
|---|---|
| SND123 | 1.87 |

TABLE 4D
IC50 values against brain carcinoma cell lines

| Cell Line/IC50 (μM) | SK-N-AS | SH-SY-5Y | CHP-134 | U-118 |
|---|---|---|---|---|
| SND123 | 3.5 | 4.9 | 1.8 | 8.3 |

FIG. 1 shows an IC50 curve of SND118 (Cpd A) against brain carcinoma cell line U-87. FIG. 2 shows an IC50 curve of SND124 (Cpd B) against brain carcinoma cell line U-87.

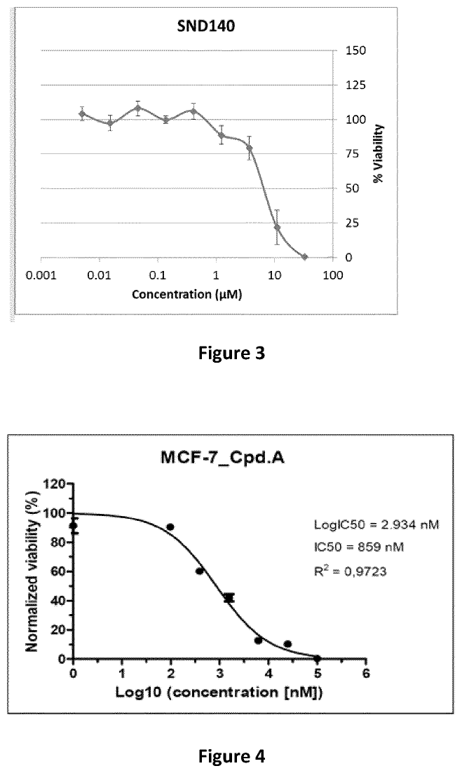

SND140 inhibited a PDX brain carcinoma with an IC50 of 6.47 μM. FIG. 3 shows an IC50 curve of SND140 against a brain carcinoma PDX GBM14-CHA.

### Example 3. Activity Against Breast Carcinoma

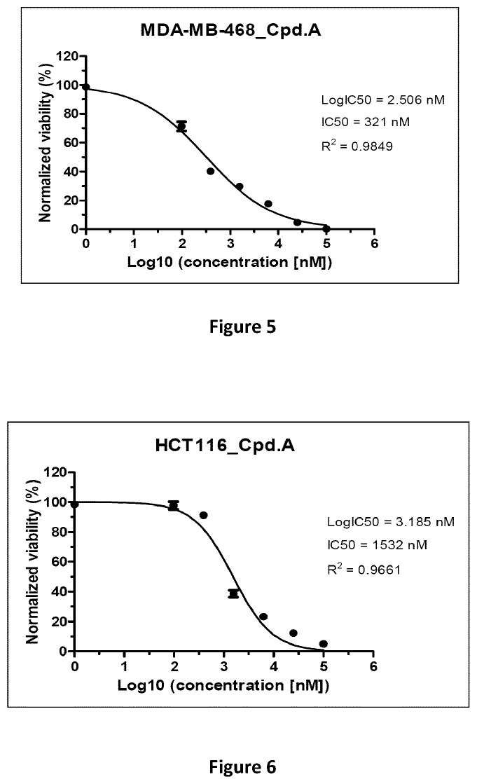

SND118, SND123, SND124, SND126A, SND127, SND140 and SND176, inhibited breast cancer cell growth with IC50s below 20 μM, as presented in Table 5A and B and in FIGS. 4 and 5.

TABLE 5A
IC50 values against breast carcinoma cell lines

| Cell Line/IC50 (μM) | MCF-7 | MDA-MB-468 |
|---|---|---|
| SND118 | 0.86 | 0.32 |
| SND123 | 6.87 | 0.36 |
| SND124 | 2 | 0.5 |
| SND126A | 12.88 | 0.56 |
| SND127 | 6.7 | 0.6 |
| SND140 | 4.3 | 2.8 |
| SND176 | 18.32 | 1.42 |

TABLE 5B
IC50 values against breast carcinoma cell lines

| Cell | | MDA- | MDA- | | | |
|---|---|---|---|---|---|---|
| Line/IC50 | BT- | MB- | MB- | | ZR- | |
| (μM) | 474 | 436 | 231 | HCC1806 | 75-1 | T47D |
| SND123 | 1.6 | 2.3 | 2 | 2.3 | 2.5 | 1.7 |

FIG. 4 shows an IC50 curve of SND118 (Cpd A) against breast carcinoma cell line MCF-7. FIG. 5 shows an IC50 curve of SND118 (Cpd A) against breast carcinoma cell line MDA-MB-468.

SND123, SND124, SND126A and SND140 inhibited the growth of breast PDX with IC50s below 20 μM as shown in Table 6.

TABLE 6
IC50 values against breast PDX

| PDX/IC50 (μM) | BR-05-0399 | BR-05-0014E |
|---|---|---|
| SND123 | 2.1 | 0.98 |
| SND124 | 5.2 | 7.2 |
| SND126A | 6.5 | 7.7 |
| SND140 | 6.0 | 10.4 |

### Example 4. Activity Against Colon Carcinoma

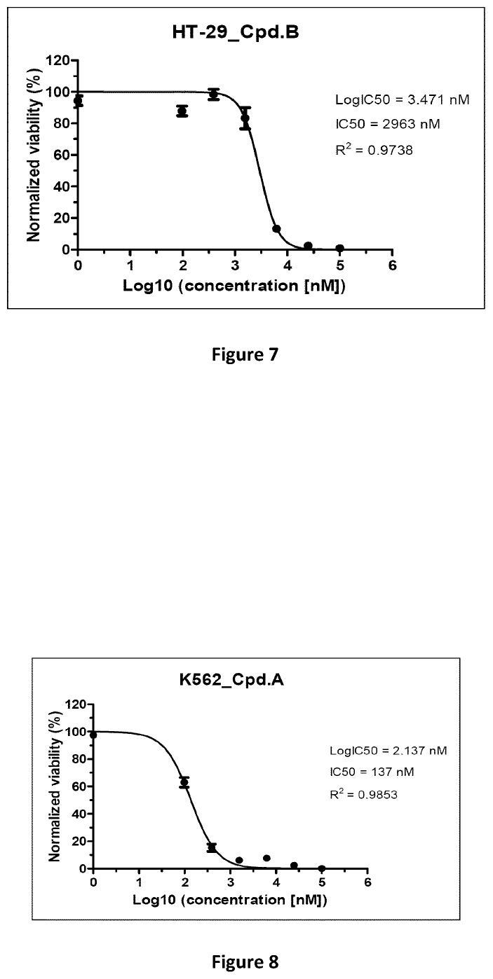

SND118, SND123, SND124, SND126A, SND127, SND140 and SND176 inhibited colon cancer cell growth with IC50s below 20 μM, as presented in Tables 7A-7D and FIGS. 6 and 7.

TABLE 7A
IC50 values against colon carcinoma cell lines

| Cell Line/IC50 (μM) | HCT-116 | LoVo |
|---|---|---|
| SND118 | 1.53 | 7.9 |
| SND123 | 2.25 | 1.70 |
| SND124 | 4.2 | 21.4 |
| SND126A | 3.56 | 2.94 |
| SND127 | 6.2 | 8.4 |
| SND140 | 10.6 | 16.3 |
| SND176 | 8.68 | 8.6 |

TABLE 7B
IC50 values against colon carcinoma cell lines

| Cell Line/IC50 (μM) | HT-29 |
|---|---|
| SND118 | 10.3 |
| SND123 | 9.30 |
| SND124 | 2.96 |
| SND127 | 6.4 |
| SND140 | 14.25 |

TABLE 7C
IC50 values against colon carcinoma cell lines

| Cell Line/IC50 (μM) | HCT-15 |
|---|---|
| SND118 | 3.3 |
| SND123 | 9.3 |
| SND124 | 10 |

TABLE 7D
IC50 values against colon carcinoma cell lines

| PDX/IC50 (μM) | KM-12 |
|---|---|
| SND123 | 3 |
| SND124 | 2.7 |
| SND126A | 3.2 |
| SND140 | 5.6 |

FIG. 6 shows an IC50 curve of SND118 (Cpd A) against colon carcinoma cell line HCT116. FIG. 7 shows an IC50 curve of SND124 (Cpd B) against colon carcinoma cell line HT-29.

SND123, SND124, SND126A and SND140 inhibited the growth of colon PDX with IC50s below 20 μM as shown in Table 8; SND124 and SND126A showed marked selectivity toward CO-04-0722 and CO-04-0700.

TABLE 8
IC50 values against colon PDX

| PDX/IC50 (μM) | CO-04-0722 | CO-04-0701 | CO-04-0700 |
|---|---|---|---|
| SND123 | 2.1 | 3.4 | 2.6 |
| SND124 | 3.2 | 21.8 | 2.3 |
| SND126A | 3.4 | >30 | 2.3 |
| SND140 | 11.6 | 13.8 | 12.8 |

### Example 5. Activity Against Endometrial Cancer

SND123, SND124, SND126A and SND140 inhibited the endometrial PDX growth with IC50s below 20 μM as presented in Table 9.

TABLE 9
IC50 values against endometrial PDX

| PDX/IC50 (μM) | EN-11-0101 |
|---|---|
| SND123 | 1.8 |
| SND124 | 9.6 |
| SND126A | 10.2 |
| SND140 | 6.2 |

### Example 6. Activity Against Esophagus PDX

SND123, SND124, SND126A and SND140 inhibited the esophagus PDX growth with IC50s below 20 μM as presented in Table 10.

TABLE 10
IC50 values against esophagus PDX

| PDX/IC50 (μM) | ES-06-0002 | ES-06-0122 |
|---|---|---|
| SND123 | 2.9 | 4.5 |
| SND124 | 5.6 | 8.5 |
| SND126A | 9.3 | 8.3 |
| SND140 | 9.66 | 13.5 |

### Example 7. Activity Against Head and Neck Cancer

SND123, SND124, SND126 and SND140 inhibited the head and neck PDX growth with IC50s below 10 μM as presented in Table 11.

TABLE 11
IC50 values against head and neck PDX

| PDX/IC50 (μM) | HN-13-0020 |
|---|---|
| SND123 | 1.5 |
| SND124 | 2 |
| SND126A | 2.7 |
| SND140 | 9.1 |

### Example 8. Activity Against Kidney Cancer

SND123, SND124, SND126A, and SND140 inhibited kidney PDX growth with IC50s below 20 μM as presented in Table 12.

TABLE 12
IC50 values against kidney PDX

| PDX/IC50 (μM) | KI-12-0062 |
|---|---|
| SND123 | 3.0 |
| SND124 | 8.5 |
| SND126A | 11.0 |
| SND140 | 16.8 |

### Example g. Activity Against Leukaemia

SND118, SND123, SND124, SND126A, SND127, SND140 and SND176 inhibited leukaemia cell growth with IC50s below 20 μM, as presented in Table 13A and 13B and FIGS. 8-10.

TABLE 13A
IC50 values against leukaemia cell lines

| Cell Line/IC50 (μM) | K-562 |
|---|---|
| SND118 | 0.14 |
| SND124 | 0.02 |
| SND127 | 1.23 |

TABLE 13B
IC50 values against leukaemia cell lines

| Cell Line/IC50 (μM) | HL-60 |
|---|---|
| SND118 | 0.29 |
| SND123 | 1.08 |
| SND124 | 0.29 |
| SND126A | 0.58 |
| SND127 | 1.54 |
| SND140 | 0.9 |
| SND176 | 1.43 |

FIG. 8 shows an IC50 curve of SND118 (Cpd A) against leukaemia cell line K-562.

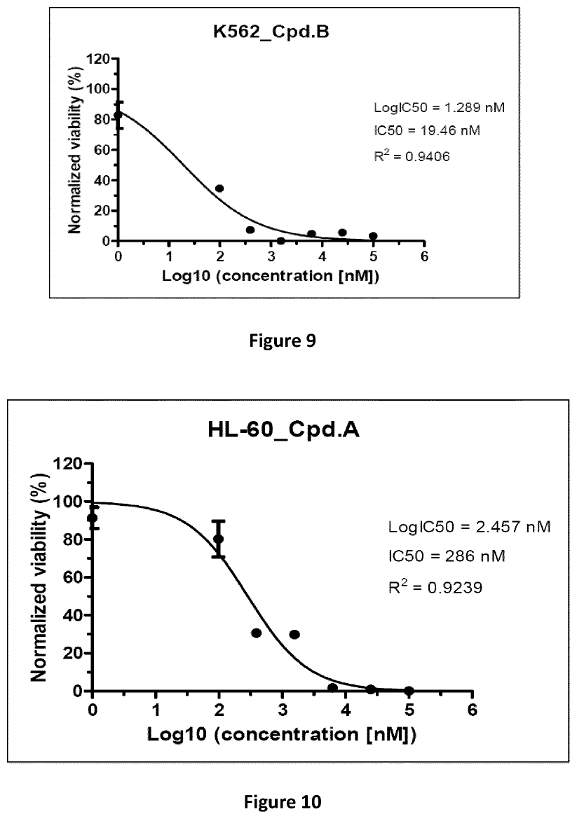

FIG. 9 shows an IC50 curve of SND124 (Cpd B) against leukaemia cell line K-562.

FIG. 10 shows an IC50 curve of SND118 (Cpd A) against leukaemia cell line HL-60.

### Example 10. Activity Against Lung Carcinoma

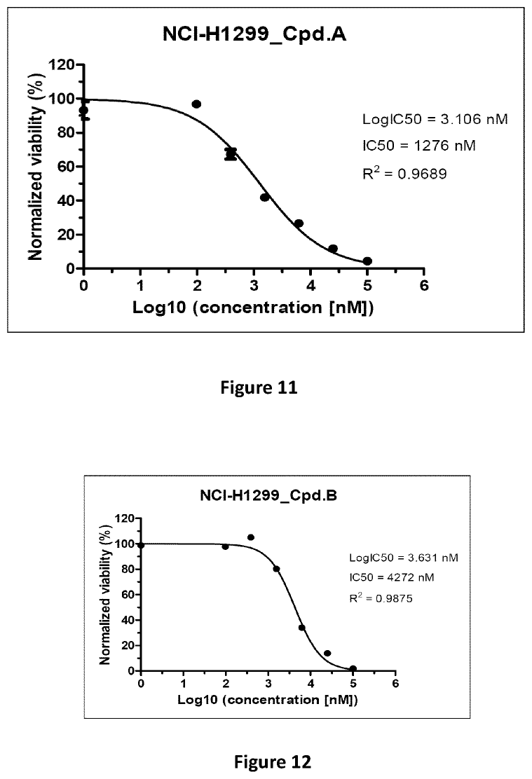

SND118, SND123, SND124, SND127 and SND140 inhibited lung carcinoma cell growth with IC50s below 20 μM, as presented in Table 14A, 14B, 14C and FIGS. 11 and 12.

TABLE 14A
IC50 values against lung carcinoma cell lines

| Cell Line/IC50 (μM) | A549 |
|---|---|
| SND118 | 12.5 |
| SND123 | 8.08 |
| SND124 | 11.2 |
| SND127 | 5.3 |

TABLE 14B
IC50 values against lung carcinoma cell line

| Cell Line/IC50 (μM) | H1299 |
|---|---|
| SND118 | 1.28 |
| SND124 | 4.27 |
| SND127 | 12.7 |

TABLE 14C
IC50 values against lung carcinoma cell lines

| Cell Line/IC50 (μM) | CALU-6 | NCI-H460 |
|---|---|---|
| SND118 | 1.47 | 0.29 |
| SND123 | 1.14 | 5.04 |
| SND127 | 7.13 | 5.58 |
| SND140 | 3.8 | 10.8 |

FIG. 11 shows IC50 curve of SND118 (Cpd A) against NSCLC cell line NCI-H-1299.

FIG. 12 shows IC50 curve of SND124 (Cpd B) against NSCLC cell line NCI-H-1299.

SND123 and SND140 inhibited growth of the SCLC doxorubicin resistant cell line H69AR, as depicted in Table 15. SND126A, SND176 exhibited a good potency against the parental H69 cells with IC50s of 3.63 μM and 7 μM respectively.

TABLE 15
IC50 values against resistant SCLC

| Cell Line/IC50 (μM) | H69AR |
|---|---|
| SND123 | 11.82 |
| SND140 | 14.6 |

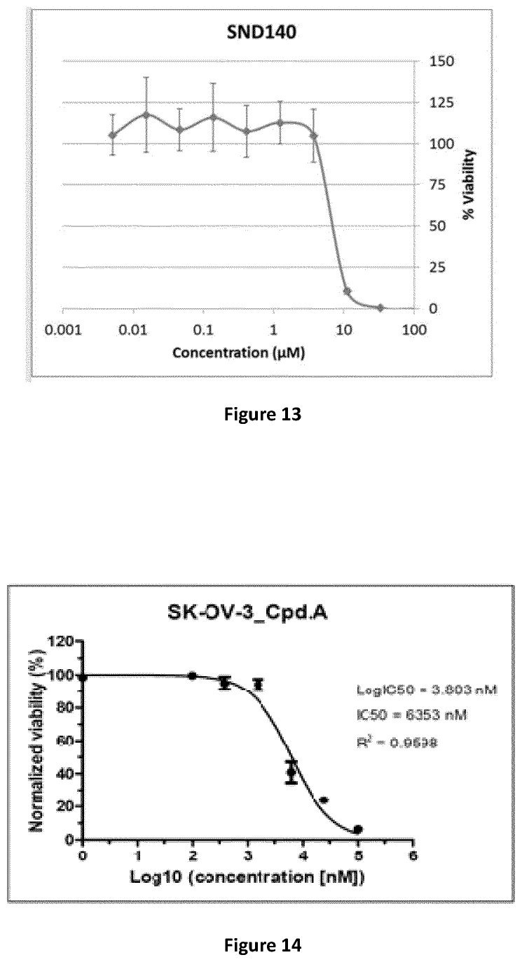

SND140 inhibited the small cell lung carcinoma PDX with an IC50 of 7.02 μM as shown in FIG. 13. FIG. 13 shows IC50 curve of SND140 against small cell lung carcinoma PDX SC6 cell line.

SND123, SND124, SND126A and SND140 inhibited the lung PDX growth with IC50s below 20 μM as presented in Table 16.

TABLE 16
IC50 values against lung PDX

| | LU-01- | LU-01- | LU-01- | LU-01- | LU-01- |
|---|---|---|---|---|---|
| PDX/IC50 (μM) | 0027 | 0010 | 0604 | 0004 | 0025 |
| SND123 | 5.9 | 0.8 | 0.35 | 3.0 | 2.4 |
| SND124 | 9.3 | 7 | 7.8 | 7.1 | 4.2 |
| SND126A | 12 | 7.6 | 8.2 | 8.4 | 5.3 |
| SND140 | 16 | 3 | 7.7 | 11.7 | 10.5 |

### Example 11. Activity Against Lymphoma

SND123, SND124, SND126A and SND140 inhibited the lymphoma PDX growth with IC50s below 5 μM as presented in Table 17.

TABLE 17
IC50 values against lymphoma PDX

| PDX/IC50 (μM) | LY-24-0340 |
|---|---|
| SND123 | 0.4 |
| SND124 | 3.7 |
| SND126A | 4.1 |
| SND140 | 4.1 |

### Example 12. Activity Against Ovarian Carcinoma

SND118, SND123, SND124, SND126A, SND127 and SND140 inhibited ovarian cancer cell growth with IC50s below 20 μM, as presented in Table 18A, 18B, 18C and FIG. 14.

TABLE 18A
IC50 values against ovarian carcinoma

| Cell Line/IC50 (μM) | SK-OV-3 |
|---|---|
| SND118 | 6.35 |
| SND124 | 3.4 |
| SND127 | 11.5 |

TABLE 18B
IC50 values against ovarian carcinoma

| Cell Line/IC50 (μM) | OVCAR-3 |
|---|---|
| SND118 | 7.3 |
| SND123 | 2.36 |
| SND126A | 6.54 |
| SND127 | 5.7 |
| SND140 | 8.87 |

TABLE 18C
IC50 values against ovarian carcinoma

| Cell Line/IC50 (μM) | OVXF 899 |
|---|---|
| SND126A | 17.6 |

FIG. 14 shows IC50 curve of SND118 (Cpd A) against ovarian cell line SK-OV-3.

### Example 13. Activity Against Pancreatic Carcinoma

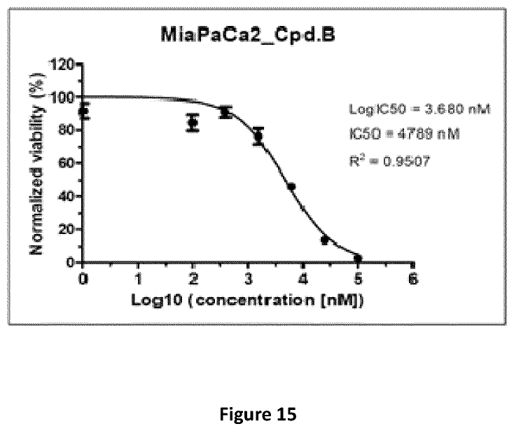

SND118, SND123, SND124, SND126A, SND127 and SND140 inhibited pancreatic cancer cell growth with IC50s below 20 μM, as presented in Table 19A and FIG. 15.

TABLE 19A
IC50 values against pancreatic carcinoma

| Cell Line/IC50 (μM) | Mia-Pa-Ca-2 |
|---|---|
| SND118 | 1.9 |
| SND123 | 1.32 |
| SND124 | 4.8 |
| SND126A | 0.96 |
| SND127 | 1.81 |

FIG. 15 shows IC50 curve of SND124 (Cpd B) against pancreatic cell lines Mia-Pa-Ca-2.

SND123 inhibited Panc-1 cell line as presented in Table 19B.

TABLE 19B
IC50 values against pancreatic carcinoma

| Cell Line/IC50 (μM) | Panc-1 |
|---|---|
| SND123 | 2.32 |

SND123, SND124, SND126A and SND140 inhibited the pancreatic PDX growth with IC50s below 20 μM as presented in Table 20.

TABLE 20
IC50 values against pancreatic PDX

| PDX/IC50 (μM) | PC-07-0045 | PC-07-0059 |
|---|---|---|
| SND123 | 4.0 | 2.7 |
| SND124 | 7.0 | 4.2 |
| SND126A | 7.7 | 4.9 |
| SND140 | 13.2 | 20.4 |

### Example 14. Activity Against Prostate Carcinoma

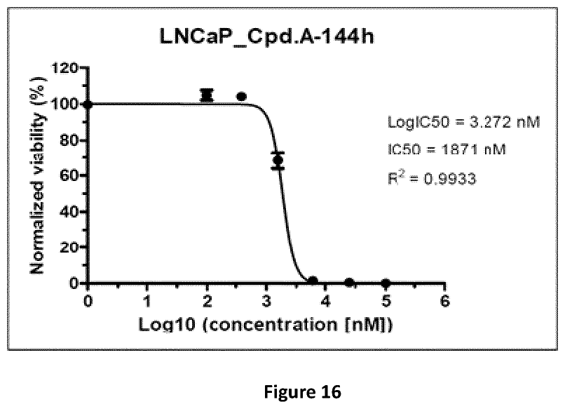

SND118, SND 123, SND124, SND126A, SND127, SND140 and SND176 inhibited prostate cancer cell growth with IC50s below 10 μM, as presented in Table 21 and FIG. 16. The data suggests that the whole class of these novel derivatives is very potent against all prostate cell lines.

TABLE 21
IC50 values against prostate carcinoma

| Cell Line/IC50 (μM) | PC-3 | LNCaP | 22Rv1 |
|---|---|---|---|
| SND118 | 5.3 | 1.87 | 5.1 |
| SND123 | 1.89 | 1.37 | 1.49 |
| SND124 | 6.3 | 4 | 3.4 |
| SND126A | 2.91 | 1.95 | 2.39 |
| SND127 | 5.5 | 2 | 3.3 |
| SND140 | 8.31 | 6.23 | 9 |
| SND176 | 7.3 | 3.66 | 8.18 |

FIG. 16 shows IC50 curve of SND118 (Cpd A) against prostate cell line LNCaP.

### Example 15. Activity Against Skin Melanoma

SND123, SND124, SND126A and SND140 inhibited the skin melanoma PDX growth with IC50s below 10 μM as presented in Table 22.

TABLE 22
IC50 values against skin melanoma PDX

| PDX/IC50 (μM) | ME-21-0028 |
|---|---|
| SND123 | 0.8 |
| SND124 | 7.7 |
| SND126A | 8 |
| SND140 | 1.1 |

### Example 16. Activity Against Stomach Cancer

SND123, SND124, SND126A and SND140 inhibited the stomach PDX growth with IC50s below 20 μM as presented in Table 23.

TABLE 23
IC50 values against stomach PDX

| | ST-02- | ST-02- | ST-02- | ST-02- |
|---|---|---|---|---|
| PDX/IC50 (μM) | 0007 | 0173 | 0012 | 0322 |
| SND123 | 1.5 | 2.2 | 0.3 | 5.2 |
| SND124 | 10.2 | 11.3 | 8.2 | 10.6 |
| SND126A | 10.7 | 14.7 | 8.9 | 9.8 |
| SND140 | 12 | 9.8 | 4.9 | 13.3 |

### Example 17. Kinase Inhibition Activity

In order to further understand if the tumour inhibition activity is due to the inhibition of certain cancer associated kinases, selected compounds were tested in the KINOMEscan™ assay against 30 kinases. SND118, SND123 and SND140 showed selective inhibitory activity against a small number of kinases as presented in Table 24.

TABLE 24
Kd values kinase inhibition

| | No. responsive | | |
|---|---|---|---|
| Cpd | kinases | Kinase name | Kd (μM) |
| SND118 | 1 | ADCK3 | 1.5 |
| SND123 | 4 | ABL1(E255K)- | 0.97 |
| | | phosphorylated | |
| | | ABL1- | 1.3 |
| | | nonphosphorylated | |
| | | ABL1-phosphorylated | 0.86 |
| | | ADCK3 | 0.82 |
| SND140 | 4 | ABL1(E255K)- | 0.66 |
| | | phosphorylated | |
| | | ABL1- | 1.5 |
| | | nonphosphorylated | |
| | | ABL1-phosphorylated | 0.7 |
| | | ADCK3 | 0.62 |

### Example M8. In Vivo Tumour Inhibition of Xenograft Leukemia Model K562

Test compounds were evaluated for the in vivo inhibition activity against the chronic myelogenous leukemia CDX in NOG mice. 1×10⁷ cells were injected s.c. into the left flank at day 0. Mice were stratified into groups of 10 mice each with a mean tumor volume of 109±35 mm³ and the treatment was administered i.p. daily. SND118 and SND140 significantly inhibited tumour growth as shown by the T/C value at day 17 after tumour transplantation (Table 25)

TABLE 25
Optimal T/C values against K562 CDX

| | | | | | Dose | Weight | Optimal |
|---|---|---|---|---|---|---|---|
| | No | Treat- | | | (mg/ | loss | T/C |
| Group | mice | ment | Route | Sequence | kg/inj) | (%) | (value) |
| A | 10 | vehicle | i.p | qd | | 2 | |
| B | 10 | SND118 | i.p | qd | 10 | 8 | 58*** |
| C | 10 | SND140 | i.p | qd | 5 | 5 | 69* |

*p < 0.05,
**p < 0.01;
***p < 0.001 compared to group A by Two-way-ANOVA

It will be understood that the present invention has been described above byway of example only. The examples are not intended to limit the scope of the invention.

Various modifications and embodiments can be made without departing from the scope and spirit of the invention, which is defined by the following claims only.

1. A compound of formula (I) for use treating or preventing cancer: 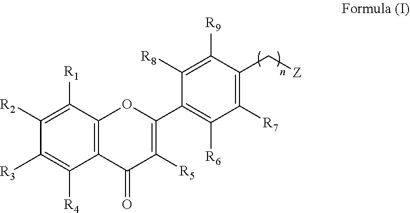
wherein:
Z is —[P(R¹¹)₃]X, wherein X is a counter anion;
R¹, and R², independently, are selected from —OH, —O—C₁₋₄ alkyl, —OC(O)R₁₃, —OC(O)NHR¹³, —OC(O)N(R¹³)₂; or
R¹ and R² together form —O—(C₁₋₃ alkylene)-O—;
R³, R⁴, R⁵, R⁶, R⁷, R⁸, and R⁹, independently, are selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(RP)₂; —NH₂; —NHR^{β}; —N(R^{β})₂; —CHO; —COR^{β}; —COOH; —COOR^{β}; —OCOR^{β}; and benzyl optionally substituted with 1-3-R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl; and
n=1-10.

2. A compound for use as claimed in claim 1, wherein the compound is a compound of Formula 1A: 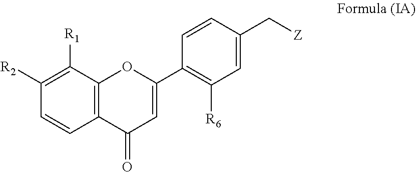
wherein:
Z is —[P(R¹¹)₃]X, wherein X is a counter anion;
R¹ and R², independently, are selected from —OH, —O—C₁₋₄ alkyl, —OC(O)R₁₃, —OC(O)NHR¹³, —OC(O)N(R¹³)₂; or
R¹ and R² together form —O—(C₁₋₃ alkylene)-O—;
R⁶ is selected from H; halo; —CN; —NO₂; —R^{β}; —OH, —OR^{β}; —SH; —SR^{β}; —SOR^{β}; —SO₂H; —SO₂R^{β}; —SO₂NH₂; —SO₂NHR^{β}; —SO₂N(RP)₂; —NH₂; —NHR^{β}; —N(R^{β})₂; —CHO; —COR^{β}; —COOH; —COOR^{β}; —OCOR^{β}; and benzyl optionally substituted with 1-3-R^{β};
each —R^{β} is independently selected from a C₁-C₆ alkyl, C₂-C₆ alkenyl, C₂-C₆ alkynyl or C₃-C₁₄ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —NO₂, —C≡CH, —CHO, —CON(CH₃)₂ or oxo (═O) groups;
each —R¹¹ is independently selected from H, C₁-C₆ alkyl, C₂-C₆ alkenyl, C₃-C₁₄ aryl group, or C₃-C₁₄ aliphatic cyclic group, and wherein any —R¹¹ may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups
each —R¹³ is independently selected from a H, C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R¹³ may optionally be substituted with one or more —R¹⁴;
each R¹⁴ is independently selected from a C₁-C₆ alkyl, C₂-C₆ alkenyl, C₂-C₆ alkynyl, C₃₋₁₄ cyclic group, halo, —NO₂, —CN, —OH, —NH₂, mercapto, formyl, carboxy, carbamoyl, C₁₋₆ alkoxy, C₁₋₆ alkylthio, —NH(C₁₋₆ alkyl), —N(C₁₋₆ alkyl)₂, C₁₋₆ alkylsulfinyl, C₁₋₆ alkylsulfonyl, or arylsulfonyl, wherein any —R₁₄ may optionally be substituted with one or more —R₁₅;
each —R¹⁵ is independently selected from halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl N-ethylcarbamoyl N,N-dimethylcarbamoyl, N,N-diethylcarbamoyl, N-methyl-N-ethylcarbamoyl, methylthio, ethylthio, methylsulfinyl, ethylsulfinyl, mesyl ethylsulfonyl, methoxycarbonyl, ethoxycarbonyl, N-methylsulfamoyl N-ethylsulfamoyl N,N-dimethylsulfamoyl N,N-diethylsulfamoyl, N-methyl-N-ethylsulfamoyl, carbocyclyl, aryl, or heterocyclyl; and
n=1-10.

3. A compound for use as claimed in claim 1 or claim 2, wherein Z is —[P(R¹¹)₃]X, wherein each —R¹¹ is independently a C₃-C₁₄ aryl group; and wherein any —R¹¹ may optionally be substituted with one or more C₁-C₄ alkyl, halo, —OH, —NH₂, —CN, —C≡CH or oxo (═O) groups.

4. A compound for use as claimed in any one or more of the preceding claims, wherein each R¹¹ is phenyl.

5. A compound for use as claimed in any one or more of the preceding claims, wherein n is 3-6, or n is 3 or 4.

6. A compound for use as claimed in any one or more of the preceding claims, wherein R³, R⁴, R⁵, R⁶, R⁷, R⁸, and R⁹, independently, are selected from H; halo; —CN; —NO₂; —R^{β}; —SH; —SR^{β}; —SOR^{β}; —SO₂H; —SO₂R^{β}; —SO₂NH₂; —SO₂NHR^{β}; —SO₂N(R^{β})₂; —NH₂; —NHR^{β}; —N(R^{β})₂; —CHO; —COR^{β}; —COOH; and —COOR^{β}; and benzyl optionally substituted with 1-3-R^{β}.

7. A compound for use as claimed in any one or more of the preceding claims, wherein R³, R⁴, R⁵, R⁶, R⁷, R⁸, and R⁹, independently, are selected from H; halo; —CN; —NO₂; —R^{β}; —NH₂; —NHR^{β}; —N(R^{β})₂; —CHO; —COR^{β}; —COOH; —COOR^{β}; and —OCOR^{β}.

8. A compound for use as claimed in any one or more of the preceding claims, wherein R³, R⁴, R⁵, R⁶, R⁷, R⁸, and R⁹, independently, are selected from H; halo; —CN; —NO₂; —R^{β}; —NH₂; —NHR^{β}; —N(R^{β})₂; and —CHO.

9. A compound for use as claimed in any one or more of claims 1 to 6, wherein R³, R⁴, R⁵, R⁶, R⁷, R⁸, and R⁹, independently, are selected from H; halo; —CN; —NO₂; —SH; —SO₂H; and —NH₂.

10. A compound for use as claimed in any one or more of the preceding claims, wherein R¹ and R², independently, are selected from —OH, —O—C₁₋₄ alkyl, —OC(O)R₁₃, and —OC(O)NHR¹³; or R¹ and R² together form —O—(C₁₋₃ alkylene)-O—.

11. A compound for use as claimed in any one or more of the preceding claims, wherein R¹ and R², independently, are selected from —OH, —O—CH₃, —OC(O)C₄-alkyl, and —OC(O)NH—C₂₋₃-alkyl; or R¹ and R² together form —O—(CH₂)—O—.

12. A compound for use as claimed in any one or more of the preceding claims, wherein R¹ and R², independently, are selected from —OH, —O—C₁₋₄ alkyl, —OC(O)R₁₃, —OC(O)NHR¹³, and —OC(O)N(R¹³)₂, or R¹ and R² together form a —O—(C₁₋₃ alkylene)-O— group; and R³, R⁴, R⁵, R⁶, R⁷, R⁸, and R⁹, independently, are selected from H; halo; —CN; —NO₂; —SH; —SO₂H; and —NH₂.

13. A compound for use as claimed in claim 1 selected from the following:

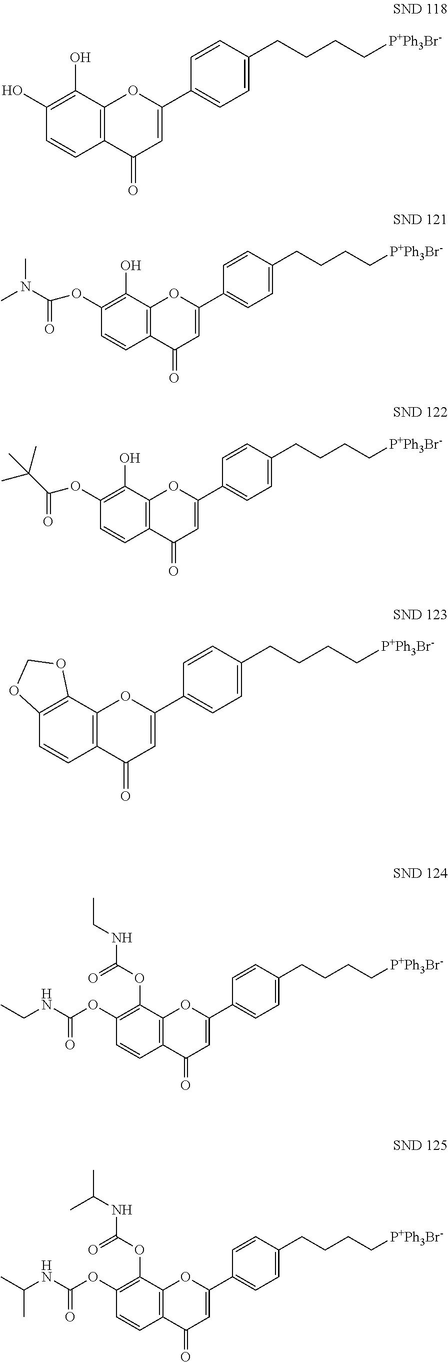

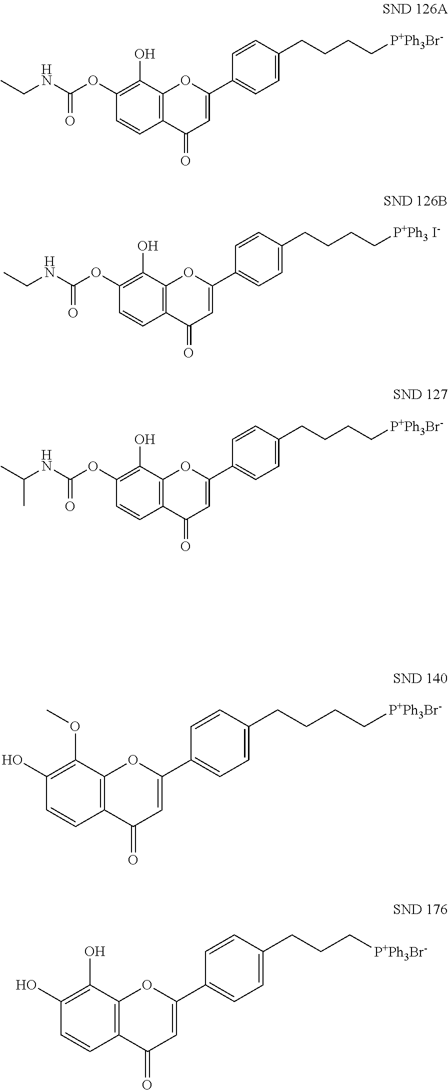

14. A compound for use according to any of claims 1 to 13, wherein the cancer is brain cancer, breast cancer, colon cancer, leukaemia, lung cancer, lymphoma, ovarian cancer, pancreatic cancer, prostate cancer, renal cancer or skin cancer (melanoma).

15. A method of treatment or prevention of cancer, the method comprising the step of administering an effective amount of a compound as defined in any one of claims 1 to 13, or a pharmaceutically acceptable multi-salt, solvate or prodrug thereof, to a subject in need thereof to thereby treat or prevent cancer.

16. A method of treatment or prevention according to claim 15, wherein the cancer is brain cancer, breast cancer, colon cancer, leukaemia, lung cancer, lymphoma, ovarian cancer, pancreatic cancer, prostate cancer, renal cancer or skin cancer (melanoma).

17. A compound selected from the group consisting of: [chemical structure]
